(19) United States
(12) Patent Application Publication
(10) Pub. No.: US 2016/0075987 A1
(43) Pub. Date: Mar. 17, 2016

(54) NOVEL 3D SCAFFOLD MICROSTRUCTURE

(71) Applicant: KING ABDULLAH UNIVERSITY OF SCIENCE AND TECHNOLOGY, Thuwal (SA)

(72) Inventors: Xixiang ZHANG, Thuwal (SA); Wei XU, Thuwal (SA); Xiaoming YANG, Thuwal (SA); Longqing CHEN, Thuwal (SA); Weijia WEN, Kowloon (HK); Ahad SYED, Thuwal (SA); Basil CHEW, Thuwal (SA); Ebtihaj M. BUKHARI, Thuwal (SA)

(73) Assignee: KING ABDULLAH UNIVERSITY OF SCIENCE AND TECHNOLOGY, Thuwal (SA)

(21) Appl. No.: 14/785,329

(22) PCT Filed: Apr. 17, 2014
(86) PCT No.: PCT/IB2014/001680

§ 371 Date: Oct. 17, 2015

Related U.S. Application Data
(60) Provisional application No. 61/812,847, filed on Apr. 17, 2013.
(60) Provisional application No. 61/909,648, filed on Nov. 27, 2013.

(51) Int. Cl. C12M 1/12; C12M 1/42

(52) U.S. Cl. CPC C12M 25/14; C12M 35/06

(57) ABSTRACT

A three-dimensional scaffold microstructure comprising a plurality of collapsed polymer columns for use in cell culture or nematode studies.

### CLAIM OF PRIORITY

This application claims priority to U.S. Provisional Patent Application No. 61/812,847, filed Apr. 17, 2013, and U.S. Provisional Patent Application No. 61/909,648, filed Nov. 27, 2013, both of which are incorporated by reference in their entirety.

### FIELD OF THE INVENTION

The invention relates to three-dimensional shaped microstructures.

### BACKGROUND

The microstructure of a material can influence the physical properties of the material. A technological need exists to better prepare metal, polymer, ceramic, and glassy structures at the micro and nanoscale including preparation of patterned structures at high resolution and registration. Polymeric microstructures are important for a wide range of applications.

### SUMMARY

In one aspect, a three-dimensional microstructure can include a plurality of collapsed polymer columns, wherein a portion of a surface of each collapsed polymer column can contact with a portion of a surface of at least another collapsed polymer column.

In some embodiments, the surface of each collapsed column can include nanoscale grooves. In some embodiments, at least two collapsed polymer columns can have substantially similar height and size dimensions when straightened. In some embodiments, at least two collapsed polymer columns can have substantially different height and size dimensions when straightened.

In some embodiments, the size of a collapsed polymer column can be less than 7 μm. In some embodiments, the size of a collapsed polymer column can be less than 5 μm. In some embodiments, the size of a collapsed polymer column can be less than 2 μm.

In some embodiments, the three-dimensional microstructure can be within a microwell. In some embodiments, each collapsed polymer column can include polydimethylsiloxane, poly(methyl methacrylate), epoxy resin, polyethylene glycol, or a copolymer thereof.

In another aspect, a device for culturing cells can include a three-dimensional microstructure, wherein the three-dimensional microstructure can include a plurality of collapsed polymer columns, wherein a portion of a surface of each collapsed polymer column can contact with a portion of a surface of at least another collapsed polymer column.

In some embodiments, the surface of each collapsed column can include nanoscale grooves. In some embodiments, at least two collapsed polymer columns can have substantially similar height and size dimensions if straightened. In some embodiments, at least two collapsed polymer columns can have substantially different height and size dimensions if straightened.

In some embodiments, the size of a collapsed polymer column can be less than 7 μm. In some embodiments, the size of a collapsed polymer column can be less than 5 μm. In some embodiments, the size of a collapsed polymer column can be less than 2 μm.

In some embodiments, the three-dimensional microstructure can be within a microwell. In some embodiments, each collapsed polymer column can include polydimethylsiloxane, poly(methyl methacrylate), epoxy resin, polyethylene glycol, or a copolymer thereof.

In some embodiments, the cells can include a HeLe cell, a SaOs-2 cell, or a NIH-3T3 cell. In some embodiments, the cells can be viable. In some embodiments, the cells can be on and within the three-dimensional microstructure. In some embodiments, the cells can be on the nanoscale grooves of the collapsed columns.

In another aspect, a method for preparing a three-dimensional microstructure can include loading a polymer onto a mold having a three-dimensional pattern including a plurality of columnar features and forming the three-dimensional microstructure by releasing the polymer from the mold, whereby the three-dimensional microstructure can arise from interfacial or capillary forces between the columnar features. In some embodiments, the polymer can include polydimethylsiloxane, poly(methyl methacrylate), epoxy resin, polyethylene glycol, or a copolymer thereof.

In some embodiments, the mold can be a silicon mold. In some embodiments, the method can include treating the silicon mold with silane to make the surface of the mold hydrophobic. In some embodiments, the mold can include a metal. In some embodiments, the mold can include a polymer. In some embodiments, the surface of the mold can be hydrophobic.

In some embodiments, each of the columnar features can comprise a pillar and a gap. In some embodiments, the size of the pillar can be less than 10 μm. In some embodiments, the size of the pillar can be less than 7 μm. In some embodiments, the size of the pillar can be less than 5 μm. In some embodiments, the size of the pillar can be less than 2 μm. In some embodiments, the width of the gap can be less than 15 μm. In some embodiments, the width of the gap can be less than 10 μm. In some embodiments, the width of the gap can be less than 7 μm. In some embodiments, the width of the gap can be less than 5 μm.

In some embodiments, the method can include etching the mold. In some embodiments, etching the mold can include a laser etching or a lithography. In some embodiments, the method can include degassing the polymer in the mold. In some embodiments, the method can include curing the polymer.

In some embodiments, the method can include releasing the polymer from the mold in an organic solvent. In some embodiments, the method can include forming the three-dimensional microstructure within a microwell.

In some embodiments, the three-dimensional microstructure can include collapsed polymer columns, wherein a partial surface of each collapsed polymer column can contact with a partial surface of at least another collapsed polymer column.

In some embodiments, at least two collapsed polymer columns can have substantially similar height and size dimensions if straightened. In some embodiments, at least two collapsed polymer columns can have substantially different height and size dimensions if straightened.

In another aspect, a method of culturing cells can include adding cells to a microwell including a three-dimensional microstructure and incubating the cells in the three-dimensional microstructure.

In some embodiments, the three-dimensional microstructure can include a plurality of collapsed polymer columns, wherein a portion of a surface of each collapsed polymer column can contact with a portion of a surface of at least another collapsed polymer column, and wherein the surface of each collapsed column can include nanoscale grooves.

In some embodiments, at least two collapsed polymer columns can have substantially similar height and size dimensions if straightened. In some embodiments, at least two collapsed polymer columns can have substantially different height and size dimensions if straightened.

In some embodiments, the cells can include HeLe cells. In some embodiments, the cells can maintain viability for at least 72 hours. In some embodiments, the cells can grow on and within the three-dimensional microstructure. In some embodiments, the cells can grow on the nanoscale grooves of the collapsed columns.

In another aspect, a three dimensional microstructure can include a plurality of polymer pillars on the surface of a substrate, wherein each polymer pillar can include a plurality of nanoparticles. In some embodiments, the polymer can include polydimethylsiloxane, poly(methyl methacrylate), epoxy resin, polyethylene glycol, or a copolymer thereof. In some embodiments, the nanoparticles can include magnetic nanoparticles. In some embodiments, the nanoparticles can include iron, cobalt, nickel, a compound thereof, or an alloy thereof. In some embodiments, the nanoparticles can include iron nanoparticles.

Different dimensions of polymer pillars can be used. In some embodiments, the size of a polymer pillar can be less than 20 μm. In some embodiments, the size of a polymer pillar can be less than 10 μm. In some embodiments, the size of a polymer pillar can be less than 5 μm. In some embodiments, the height of a polymer pillar can be less than 100 μm. In some embodiments, the height of a polymer pillar can be less than 50 μm. In some embodiments, the height of a polymer pillar can be less than 20 μm.

In another aspect, a device can include a plurality of polymer pillars, wherein each polymer pillar can include magnetic nanoparticles, and a magnetic field source that can deform the polymer pillars when applied to the pillars. In some embodiments, the magnetic field source can operate at a range of magnetic field strength. In some embodiments, the deformation of the polymer pillars can provide a mechanical stimulation to a subject on the polymer pillar. In some embodiments, the subject can be a worm, or a cell.

In another aspect, a method for studying mechanism of a subject can include applying a magnetic field to a plurality of polymer pillars, wherein each polymer pillar can include magnetic nanoparticles, and wherein the subject can contact with a portion of a surface of at least one polymer pillar.

In another aspect, a device for mixing liquid can include a Y junction microfluidic channel wherein at least two different kinds of liquid can be separately inserted into the channel, a plurality of polymer pillars within the channel, wherein each polymer pillar can include magnetic nanoparticles, and a magnetic field that can operate at a range of magnetic field strength, wherein the liquid can be mixed by applying a magnetic field to the polymer pillars.

In another aspect, a method for preparing a three-dimensional microstructure can include loading an organic solvent containing magnetic nanoparticles onto a mold having a three-dimensional pattern including a plurality of columnar features, applying a magnetic field to the mold to move the nanoparticles into the three-dimensional pattern of the mold, removing at least 70% of the organic solvent from the three-dimensional pattern of mold, loading a polymer onto the mold, and forming the three dimensional microstructure by releasing the polymer from the mold, wherein the three dimensional microstructure can include polymer pillars containing magnetic nanoparticles.

In some embodiments, a circling magnetic field can be used around the center of the microstructure pattern. In some embodiments, the magnetic force can be moved out of the area of the three-dimensional pattern to remove excess magnetic nanoparticles.

In some embodiments, the polymer in the mold can be degassed. In some embodiments, the polymer can be cured. In some embodiments, the surface of the mold can be hydrophobic. In some embodiments, the polymer can include polydimethylsiloxane, poly(methyl methacrylate), epoxy resin, polyethylene glycol, or a copolymer thereof. In some embodiments, the mold can be a silicon mold. In some embodiments, the silicon mold can be treated with silane to make the surface of the mold hydrophobic.

Other aspects, embodiments, and features will be apparent from the following description, the drawings, and the claims.

### BRIEF DESCRIPTION OF THE DRAWINGS

FIG. 1 is a series of schematic drawings illustrating the fabrication process of the three-dimensional (3D) polymer microstructure. FIG. 1(a) shows the etching of a silicon mold by standard UV lithography process; FIG. 1(b) shows the structure of a mold, FIG. 1(c) shows that polydimethylsiloxane (PDMS) polymer was loaded onto the mold; FIG. 1(d) shows that the PDMS microstructures can collapse and touch each other due to the interfacial/capillary force; In FIG. 1, black color indicates chrome mask; grey color indicates silicon wafer; and blue color indicates PDMS.

FIG. 2(a) is a scanning electron microscope (SEM) image of PDMS 3D microstructures in a microwell array; FIG. 2(b) is an SEM image of detailed PDMS microstructure inside a single microwell.

FIG. 3 shows various 3D structures formed by different molds. FIG. 3(a) is an SEM image of PDMS 3D microstructures formed by a mold with 2 μm size 10 μm gap; FIG. 3(b) is an SEM image of PDMS 3D microstructures formed by a mold with 2 μm size 5 μm gap; FIG. 3(c) is an SEM image of PDMS 3D microstructures formed by a mold with 5 μm size 7 μm gap; FIG. 3(d) is an SEM image of PDMS 3D microstructures formed by a mold with 5 μm size 10 μm gap; FIG. 3(e) is an SEM image of PDMS 3D microstructures formed by a mold with 5 μm size 5 μm gap; FIG. 3(f) is an SEM image of PDMS 3D microstructures formed by a mold with 7 μm size 10 μm gap; FIG. 3(g) is an SEM image of PDMS 3D microstructures formed by a mold with 7 μm size 7 μm gap; FIG. 3(h) is an SEM image of PDMS 3D microstructures formed by a mold with 7 μm size 5 μm gap; FIG. 3(i) is a detailed SEM image of PDMS 3D microstructures formed by a mold with 2 μm size and 5 μm gap inside a single microwell.

FIGS. 4(a) to 4 (f) are transmitted light and fluorescence superimposed images of cell colonies growing on 3D microstructures.

FIGS. 5(g) to 5(r) are SEM images of cell colonies on 3D microstructures in a single microwell; FIGS. 5(k), 5(i), 5(n), 5(p), and 5(r) are SEM images of enlarged view of the cells inside the microwell and attached to the 3D pillar microstructure.

FIG. 6(a) shows the etching of a mold; FIG. 6(b) shows treating the mold to make surface hydrophobic; FIG. 6(c) shows loading nanoparticles in organic solvent onto the mold with a magnet on the back side of the mold; FIG. 6(d) shows drying the mold in air; FIG. 6(e) shows loading polymer onto the mold; and FIG. 6(f) shows a three-dimensional microstructure with polymer pillars containing nanoparticles.

FIG. 7(a) shows the circling of a magnet close to the back side of the mold; and FIG. 7(b) shows that when the magnet was moved out of the microstructure region, excess nanoparticles were removed out of the microstructure region with the movement of the magnet.

FIG. 8(a) shows a microscope image of polymer pillar array; FIG. 8(b) shows a microscope image of polymer pillar array filled with nanoparticles; FIG. 8(c) shows a microscope image of the side view of scratched down pillars; FIG. 8(d) shows scanning electron microscope (SEM) image of a polymer pillar array.

FIG. 9(a) shows a microscope image of polymer pillar array with no magnet applied; FIG. 9(b) shows a microscope image of polymer pillar array when magnet was applied to the top right of the pillar array; and FIG. 9(c) shows a microscope image of polymer pillar array when magnet was applied to the top left of the pillar array.

FIG. 10 is a graph showing the displacement of pillars with different size of the pillars.

FIG. 11(a) shows an Energy-dispersive X-ray Spectroscopy (EDX) measurement of polymer pillars; FIG. 11(b) shows an EDX measurement of polymer pillars with iron (Fe) nanoparticles.

FIG. 12(a) is a schematic drawing of loading nematode onto Fe nanoparticles filled pillar array; and FIG. 12(b) is a schematic drawing of pillars deformed with the lateral movement of an external magnet.

FIG. 13(a) is a schematic drawing of loading cells onto a pillar array; FIG. 13(b) is a schematic drawing showing that cell spread on and adhesion to the top of the pillars cause the deformation of the underneath pillars; FIG. 13(c) is a schematic drawing showing that when a magnetic force is applied, the deformation of the pillars underneath the cells is reduced; FIG. 13(d) is a schematic drawing showing that when a strong magnetic force is applied, the deformation of the pillars underneath the cells is prevented.

FIG. 14(a) is a schematic drawing showing that when two liquids are injected into a Y junction microfluidic channel, if no external magnetic force is exerted, liquid/liquid interface is observed in the downstream of the Y junction channel; and FIG. 14(b) is a schematic drawing showing that with the application of external magnetic force, the pillars can move or swirl vertically to the flow direction and thus cause mixture of two liquid in the downstream of the Y junction channel.

FIG. 15 shows fabrication process flow of the PDMS 3D microstructures(A) and SEM image of the top view (B) and side view (C) of the fabricated PDMS 3D microstructures, micropillar size 5 μm; the arrows in (C) are pointing at three types of collapsed micropillars: tilted micropillar (white arrow), bowed micropillar (red arrow) and curved micropillar (green arrow). Scale bar is 10 μm.

FIG. 16 shows top view (top position) and side view (bottom position) SEM pictures of 3D microstructures formed by various dimension micropillars. FIG. 16(A) shows 3D microstructure of 5 μm size and 5 μm gap micropillar and the length of 35 μm, 45 μm, 50 μm and 55 μm (from left to right); FIG. 16(B) shows average height of 3D microstructure formed by collapsed micropillars with varied gap and FIG. 16(C) shows average height of 3D microstructure formed by collapsed micropillars with varied size. Scale bar is 20 μm.

FIG. 17 shows SEM images of the hybrid 3D microstructrues of 5 μm size micropillar (A) and 3 μm size micropillar (B). In both parts, low magnification view picture is on the left and the enlarged view of nanostructure is on the right.

FIG. 18 shows stained cytoskeleton (red) and focal adhesion (green) confocal images; the nucleus was stained with DAPI (blue color); (A) SaOs-2 cells on unpatterned flat PDMS surface (left) and 3D microstructure device (right); (B) NIH-3T3 cells on unpatterned flat PDMS surface (left) and 3D microstructure device (right); (C) 3D surface rendering of SaOs-2 cells and NIH-3T3 cells, which demonstrated that both cells either followed the topography of the 3D microstructures pattern or penetrated into the gap region and formed a complicated 3D morphology; (D) focal adhesion immunofluorescence confocal slice image, which revealed nice alignment of SaOs-2 and NIH-3T3 to the geometry of side/top of micropillars. Pointed by white arrows. Scale bar is 20 μm.

FIG. 19 shows SEM images of false colored (blue) SaOs-2 cells (A) and NIH-3T3 (B) cells proliferated on 3D microstructures, which showed that cells can follow the topology of the 3D microstructure and cover multiple micropillars (left). When they met at the gap region, they can form connection to penetrate the gap region or find the adhesion position within the gap region (middle). On 10 μm gap 3D microstructures, where large area of substrate was exposed, a main body of cells were not observed to adhere only on the substrate. Cells main body remained adhered to the micropillars and formed branches to the substrate (right). Scale bar is 10 μm. FIG. 20 shows surface rendering confocal images of SaOs-2 cells(A) and NIH-3t3 cells (B) nucleus on flat unpatterned surface (left) and 3D microstructure device (middle), which demonstrated the deformation of cells nuclei. The superimposed confocal transmitted light image and DAPI image also showed that the deformation of the nucleus followed the topography of the 3D microstructures. Scale bar is 7 μm.

FIG. 21(A) shows the confocal slice image that revealed the branches of SaOs-2 (left) and NIH-3T3 (right) cells containing clear F-actin filaments (pointed by white arrows); FIG. 21(B) shows superimposed transmitted light and fluorescence confocal image of SaOs-2 (left) and NIH-3T3 (right) cells demonstrating the nice alignment of F.A. to the collapsed micropillars (pointed by white arrows). Scale bar is 10 μm.

FIG. 22 shows superimposed transmitted light and fluorescence confocal images of SaOs-2 (left) and NIH-3T3 (right) cells demonstrating the deformation of the nucleus (stained with DAPI) of cells cultured on 3D microstructures (pointed by white arrows). Scale bar is 10 μm.

FIG. 23 shows images of SaOs-2 cells cultured on the hybrid 3D microstructures. FIG. 23(A) shows confocal MIP image of SaOs-2 cell F-actin and F.A.; FIG. 23(B) are surface 3D rendering image showing the 3D morphology of SaOs-2 cells; FIG. 23(C) shows superimposed transmitted light and fluorescence confocal image of SaOs-2 cells demonstrating the alignment of F.A. to the collapsed hybrid micropillars (pointed by white arrows); FIG. 23(D) shows false colored SEM image demonstrating the morphology of SaOs-2 cells (labeled with blue color) cultured on the hybrid 3D microstructures. Scale bar is 10 μm.

FIG. 24 shows deformation of SaOs-2 cell nuclei when cultured on the hybrid 3D microstructures. FIG. 24(A) are 3D surface rendering confocal images showing the deformed and 3D distributed nuclei of SaOs-2 cells; FIG. 24(B) shows superimposed transmitted light and fluorescence confocal slice image demonstrating the deformation of the SaOs-2 cell nucleus (pointed by white arrow). Scale bar is 10 μm.

### DETAILED DESCRIPTION

The ability to fabricate artificial three-dimensional (3D) extracellular microenvironments can be critical for in-vitro device to mimic the extracellular matrix microenvironment of the native tissues. The fabrication of complex 3D microstructures based on Poly(dimethylsiloxane) (PDMS), a well-known and widely used bio-microelectromechanical systems (bio-MEMS) material, remains a challenge. By utilizing the instability of high aspect ratio (HAR) micropillars, PDMS based self-assembled 3D complex extracellular environment can be generated. The fabrication can be a simple single step casting replication process, which makes the fabrication process achievable at most research labs. The dependence of the formation of the PDMS 3D microstructures on the geometric parameters can been investigated. Cell lines, such as SaOs-2 and NIH-3T3 cell lines, can be cultured on the fabricated microdevices and can have strong interaction to the 3D microstructure, which indicate the strong potential of the proposed approach for chip-based cell research.

The 3D microstructures have been intensively studied recently for tissue engineering and cell culture because the two dimensional (2D) cell culture system is not able to mimic the three dimensional in-vivo extracellular matrix environment (ECM). See, for example, Carletti, E. et al., Methods Mol Biol 695, 17-39, doi:10.1007/978-1-60761-984-0_{—}2 (2011); Owen, S. C. et al., J Biomed Mater Res A 94, 1321-1331, doi:10.1002/jbm.a.32834 (2010); Nikkhah, M. et al., Biomaterials 33, 5230-5246, doi:10.1016/j.biomaterials.2012.03.079 (2012); Rimann, M. et al., Curr Opin Biotechnol 23, 803-809, doi:10.1016/j.copbio.2012.01.011 (2012); Justice, B. A. et al., Drug Discov Today 14, 102-107, doi:10.1016/j.drudis.2008.11.006 (2009); Pampaloni, F. et al., Nat Rev Mol Cell Biol 8, 839-845, doi:10.1038/nrm2236 (2007), each of which is incorporated by reference in its entirety. Cells can mirror their behaviors in the real tissues such as cell-cell interaction and cell-ECM interaction, when cultured inside the artificial 3D extracellular microenvironment. See, for example, Schmeichel, K. L. et al., J Cell Sci 116, 2377-2388, doi:10.1242/jcs.00503 (2003); Albrecht, D. R. et al., Nat Methods 3, 369-375, doi:10.1038/nmeth873 (2006); Abbott, A., Nature 424, 870-872, doi:10.1038/424870a (2003), each of which is incorporated by reference in its entirety. Therefore, it is essential to develop 3D microstructures by which an in-vivo like cell culture environment can be provided and manipulated to perform fundamental biological cell studies.

Currently, various techniques based on different materials have been applied to create 3D microstrucure, including collagen gels or hydrogels 3D scaffolds, polymer nanofibers layer by electrospinning, porous structure by phase separation or porogen leaching, 3D nanostructures by corner lithography, complex porous scaffolds by stereo-lithography and three dimensional microstructured scaffolds by direct laser writing (DLW) technique. See, for example, East, E. et al., Journal of Tissue Engineering and Regenerative Medicine 3, 634-646 (2009); Hwang, C. M. et al., Biofabrication 2 (2010); Kumar, G. et al., Biomaterials 32, 9188-9196 (2011); Mauck, R. L. et al., Tissue Engineering Part B-Reviews 15, 171-193 (2009); Jiang, L. et al., Adv Mater 24, 2191-2195 (2012); Stevens, M. M., Biophys J 100, 189-189 (2011); Hsu, S. H. et al., Acta Biomater, doi:10.1016/j.actbio.2013.02.012 (2013); Stevens, M. M. et al., Science 310, 1135-1138 (2005); Mao, J. F. et al., Materials Science & Engineering C-Materials for Biological Applications 32, 1407-1414 (2012); Berenschot, E. J. W. et al., Small 8, 3823-3831 (2012); Berenschot, E. et al., 3rd IEEE International Conference on Nano/Micro Engineered and Molecular Systems, NEMS 2008, Jan. 6, 2008-Jan. 9, 2008. 729-732 (Inst. of Elec. and Elec. Eng. Computer Society); Gauvin, R. et al., Biomaterials 33, 3824-3834 (2012); Zhang, A. P. et al., Adv Mater 24, 4266-+(2012); Soman, P. et al., Biomedical Microdevices 14, 829-838 (2012); Klein, F. et al., Adv Mater 22, 868-+(2010); Greiner, A. M. et al., Macromolecular Bioscience 12, 1301-1314 (2012), each of which is incorporated by reference in its entirety. Besides these approaches, 3D microstructures generated based on Poly(dimethylsiloxane) (PDMS) are of particular interest because PDMS elastomer is a widely used bio-MEMS material. PDMS has advantages such as excellent optical transparency, low autofluorescence, biocompatibility, ease of fabrication and a large number of successful surface modification techniques available in the literature. See, for example, Sia, S. K. et al., Electrophoresis 24, 3563-3576 (2003); Piruska, A. et al., Lab on a Chip 5, 1348-1354 (2005); Thangawng, A. L. et al., Biomedical Microdevices 9, 587-595 (2007); Zhou, J. W. et al., Electrophoresis 31, 2-16 (2010); Almutairi, Z. et al., Colloids and Surfaces a-Physicochemical and Engineering Aspects 415, 406-412 (2012); Moon, M. W. et al., Scripta Materialia 60, 44-47 (2009); Hattori, K. et al., Biotechnology Journal 5, 463-469 (2010); Regehr, K. J. et al., Lab on a Chip 9, 2132-2139 (2009); Park, J. Y. et al., Sensors and Actuators B-Chemical 173, 765-771 (2012), each of which is incorporated by reference in its entirety.

However, despite the above advantages, many current PDMS 3D microstructures for cell research are limited in the simple forms such as micropillar array or array of microwell with different shapes. See, for example, Yang, M. T. et al., Adv Mater 19, 3119-+, doi:DOI 10.1002/adma.200701956 (2007); Ochsner, M. et al., Lab on a Chip 7, 1074-1077 (2007); Xu, Y. C. et al., Biomicrofluidics 6 (2012), each of which is incorporated by reference in its entirety. Complex PDMS 3D structures can be prepared by aligning and bonding multiple layer of PDMS or by replicating mold with complex 3D structures. See, for example, Zhang, M. Y. et al., Lab on a Chip 10, 1199-1203 (2010); Karlsson, J. M. et al., Journal of Micromechanics and Microengineering 22 (2012); Schaap, A. et al., OPTICAL MATERIALS EXPRESS 3, 9 (2013); LaFratta, C. N. et al, J Phys Chem B 108, 11256-11258, doi:Doi 10.1021/Jp048525r (2004), each of which is incorporated by reference in its entirety. Such complex structures allow more modification of the in-vitro 3D microenvironment and therefore can better reveal fundamental cellular process such as adhesion, migration, proliferation, differentiation and so on. However, when fabricating these complex PDMS device, the challenging multilayer alignment and bonding process are normally involved. In addition, the fabrication of the complex 3D structured mold is not easy at large scale fabrication is sensitive in MEMS. Therefore, it is important to simplify the fabrication of PDMS complex 3D microstructrures for generating artificial 3D extracellular microenvironment.

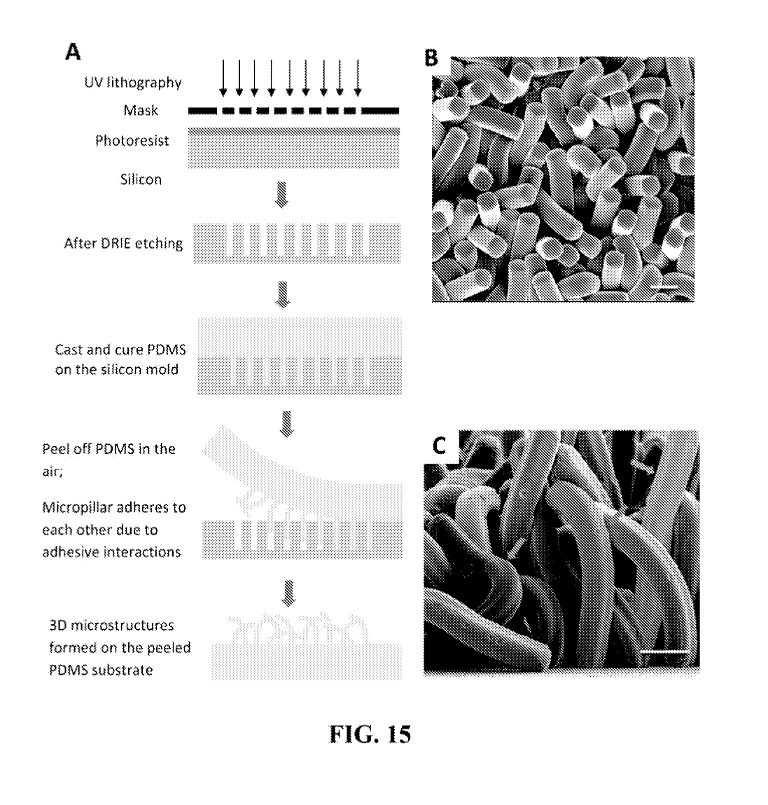

Self-assembled complex PDMS 3D topographical micropatterns can be easily and quickly fabricated to create an artificial 3D extracellular microenvironment for cell biological study. The fabrication process can be seen in FIG. 15(A). The HAR PDMS micropillars can be instable. See, for example, Chandra, D. et al., Accounts of Chemical Research 43, 1080-1091 (2010); Roca-Cusachs, P. et al., Langmuir 21, 5542-5548 (2005), each of which is incorporated by reference in its entirety. The micropillars can collapse and thus self-assembly form a complex 3D microstruture when the PDMS layer is peeled from the mold in the air. The formation process can finish right after the peeling. In addition, the mold for the fabrication of such complex 3D microstructures can be an array of HAR microholes which can easily be fabricated by standard deep reactive ion etching (DRIE) process and is achievable at most research labs. The effect of dimensional parameters, such as the spacing at gap, pitch, spacing, length and width of micropillars on the formation of the 3D microstructures, can be investigated. By tuning these parameters, different forms of 3D microstructures can be obtained. Moreover, nanostructures can even be integrated into the PDMS 3D microstructures by simply tuning the mold etching recipe without increasing the complexity of mold fabricating process. The culture of cell lines, such as SaOs-2 and NIH-3T3 cell lines, can be studied on the device, and these 3D PDMS microstructures can be very useful platform for cell research.

In addition, the PDMS 3D microstructures differ from other reported work utilizing instability of HAR micropillar array to generate 3D microstructures. See, for example, Pokroy, B. et al., Science 323, 237-240 (2009), which is incorporated by reference in its entirety. Those works have been focused mainly on utilizing the liquid capillary force to create the micropillar lateral collapse. Consequently, the resulted 3D microstructures were segregated micropillars cluster array, not the continuous 3D structures. By contrast, using a method described here, continuous microstructures can be generated over a large area without the need of using any type of liquid.

As an inexpensive, simple alternative to conventional lithography, a method using interfacial or capillary forces provides new possibilities to create polymer microstructures. A method of preparing a polymer microstructure can include loading the liquid polymer onto a mold with microstructure/nanostructure patterns, curing the polymer, releasing the polymer layer from the mold. Polymer microstructures can be prepared in a microwell array. In another embodiment, polymer microstructures can be prepared on a surface.

The mold can have a three-dimensional pattern including a plurality of columnar features. The microstructures on the released polymer layer can collapse to the substrate or adhere to each other to form three-dimensional structures if the microstructures dimension and shape are properly designed. A portion of a surface of each collapsed polymer column can contact with a portion of a surface of at least another collapsed polymer column, and the surface of each collapsed column includes nanoscale grooves. Each collapsed polymer column can have substantially similar height and size dimensions if straightened. Alternatively, at least two collapsed polymer columns can have substantially different height and size dimensions if straightened.

During the process, no complicated multilayer mask lithography or chemical etching process needs to be involved. The releasing of the polymer can also be achieved in an organic solvent, such as ethanol. Polymer microstructures can be prepared using (polydimethylsiloxane) PDMS, polystyrene, PMMA, epoxy resin, poly(ethylene glycol) (PEG), poly(ethylene glycol) diacrylates, dimethacrylates, tetraacrylates, a copolymer thereof, and so on. Microstructures prepared using capillary or interfacial force can be used in microelectromechanical systems, biomedicine, biomedical device and nanotechnology.

The polymer structures can be used to culture cells, for examples, by adding cells to a microwell including a three-dimensional microstructure and incubating the cells in the three-dimensional microstructure. In a different embodiment, cells are added to and incubated in a three-dimensional microstructure on a surface. The cells can maintain viability for at least 72 hours. The cells can grow on and within the three-dimensional microstructure. The cells can grow on the nanoscale grooves of the collapsed columns.

A three dimensional microstructure can include a plurality of polymer pillars on the surface of a substrate, wherein each polymer pillar can include a plurality of nanoparticles. The nanoparticles can include magnetic material, such as iron, cobalt, nickel, or a compound thereof, or an alloy thereof. When a magnetic field is applied to the polymer pillars, the polymer pillars can deform and the degree of deformation can depend on the strength of the magnetic field.

A plurality of polymer pillars, wherein each polymer pillar includes magnetic nanoparticles, can be used in a device. When including a magnetic field source that can deform the polymer pillars, the device can be used in many applications, such as studying the movement of a subject (for example, a cell or a nematode), in a microfluidic micromixer, and so on.

A method for preparing a plurality of polymer pillars can include loading an organic solvent containing magnetic nanoparticles onto a mold having a three-dimensional pattern, applying a magnetic field to the mold to move the nanoparticles into the three-dimensional pattern of the mold, removing the organic solvent from the three-dimensional pattern of mold, loading a polymer onto the mold, and forming the three dimensional microstructure by releasing the polymer from the mold.

### Microstructures and its Preparation

Microstructure is the structure that can be revealed or made visible by a microscope. Herein microstructure is defined as structures having features in the size range of about 10 nm to about 1000 μm. The microstructure of a material can strongly influence physical properties such as strength, toughness, ductility, hardness, corrosion resistance, high/low temperature behavior, wear resistance, and so on, which in turn govern the application of these materials in industrial practice. Microstructure can be formed using a variety of materials, such as metallic, polymeric, ceramic, or composite material.

A technological need exists to better prepare metal, polymer, ceramic, and glassy structures at the micro and nanoscale including preparation of patterned structures at high resolution and registration. For example, patterned polymeric structures have attracted significant interest for applications in the development of, for example, sensors, catalysis, and optical devices. Thus far, several strategies, based upon photolithography, electron beam lithography, and micro-contact printing have been developed for generating polymer arrays. While useful for some applications, these methods are complex. A general need exists to be able to combine high registration, high resolution patterning with microscopic and nanoscopic control of polymerization, including formation of polymer brushes.

The formation of patterned structures on micrometer-length scales is essential for the fabrication of many electronic, optical and mechanical devices. Patterning technologies are well established for semiconductors and metals, but are relatively undeveloped for organic polymers. Techniques for forming microstructures in structural and functional polymers would allow micro fabrication of a broader range of organic materials.

Patterned polymer structures are of increasing importance especially for array-based platforms because of their ability to modify surface properties and their potential applications in surface-based technologies, such as protein-resistant coatings, switchable sensors, substrates for cell-growth control, and for the separation of biological molecules. Polymeric microstructures are important for a wide range of applications, including microelectromechanical systems, biomaterials, drug delivery, self-assembly, and other applications. The ability to controllably synthesize such microstructures is increasingly significant for enabling applications such as paints, rheological fluids, catalysis, diagnostics, and photonic materials.

Polymer microstructure synthesis can be carried out using processes such as photolithography, stamping, emulsion polymerization, or by an emulsion-based microfluidic technique such as flow-through microfluidic synthesis.

Microstructures can be prepared with photolithography. For example, to prepare poly(ethylene glycol) hydrogel microstructures, a silicon/silicon dioxide surface can be treated with 3-(trichlorosilyl)propyl methacrylate to form a self-assembled monolayer with pendant acrylate groups. A solution containing an acrylated or methacrylated poly(ethylene glycol) derivative and a photoinitiator (2,2-dimethoxy-2-phenylacetophenone) can then be spin-coated onto the treated substrate, exposed to 365 nm ultraviolet light through a photomask, and developed with either toluene, water, or supercritical CO₂. As a result of this process, three-dimensional, cross-linked poly(ethylene glycol) hydrogel microstructures can be immobilized on the surface.

Polymer nanostructures, such as polymer brushes, can be prepared using direct-write nanolithography by patterning a first nanostructure of a polymerizable compound on a substrate, adding polymerization catalyst to the nanostructure of polymerizable compound to form a second nanostructure which can initiate the polymerization reaction, and polymerizing monomer on the second nanostructure to form a third polymeric nanostructure.

Polymer microstructures can be prepared using laser ablation. For example, femtosecond laser ablation with near IR wavelength can be employed to fabricate microstructures on Poly(methyl methacrylate) (PMMA) substrates. In order to adjust the pulse energy of a laser beam, a linearly polarized Gaussian laser beam can be first attenuated by a rotatable half-wave plate and a polarization beam splitter. One portion of the laser beam can be split off with a beam splitter and the deflected beam can be directed into a power detector to measure the pulse energy of the laser beam. The transmission laser beam can be passed through a mechanical shutter and a series of reflective mirrors system. Finally, the transmission beam can enter a microscope objective lens and can be focused along the normal direction on the surface of the PMMA substrate to prepare polymer microstructures.

Although these techniques have provided significant advances in microstructure synthesis, it is found in general that each limits microstructure composition and/or geometry. For example, photolithographic techniques generally limit the microstructure material to that which is compatible with a photolithographic process, e.g., requiring a photoresist as the structural material. Historically, the synthesis of polymeric microstructures with microfluidics has focused almost exclusively on spheroidal microstructures, in part because the minimization of microstructure interfacial energy leads to the formation of spheres or deformations of spheres such as rods, ellipsoids or discs, or cylinders. These methods are also complex and expensive.

As an inexpensive alternative to conventional lithography, a method using interfacial or capillary forces provides new possibilities to create polymer microstructures. A method of preparing a polymer microstructure can include loading the liquid polymer onto a mold with microstructure/nanostructure patterns, curing the polymer, releasing the polymer layer from the mold. The microstructures on the released polymer layer will collapse to the substrate or adhere to each other to form 3D structures if the microstructures dimension and shape are properly designed. Comparing with other methods, this method simplifies the fabrication process of 3D microstructures.

Capillary attraction, or capillarity, is the ability of a material to move in narrow spaces without the assistance of, and in opposition to external forces like gravity. The effect can be seen in the drawing up of liquids between the hairs of a paint-brush, in a thin tube, in porous materials such as paper, in some non-porous materials such as liquified carbon fiber, or in a cell. It occurs because of inter-molecular attractive forces between surfaces.

In one embodiment, preparing a three-dimensional microstructure can include loading a polymer onto a mold having a three-dimensional pattern including a plurality of columnar features and forming the three-dimensional microstructure by releasing the polymer from the mold, whereby the three-dimensional microstructure arises from interfacial or capillary forces between the columnar features. Microstructures prepared using capillary or interfacial force can be used in microelectromechanical systems, biomedicine, biomedical device and nanotechnology.

Each of the columnar features can comprise a pillar and a gap. A pillar in the mold is a feature, such as a hole, that can be loaded with polymer. A gap in the mold is a feature that will create space between polymer pillars after polymer is released from the mold. Pillar size is the diameter of a pillar. For a square shape pillar, pillar size refers to the length of each side of the cross section of the pillar. Gap is the feature between the edges of two adjacent pillars. The width of a gap in the mold is the distance between the edges of two adjacent pillars. The size of the pillar can be varied from 1 μm to 10 μm. The size of the pillar can be less than 10 μm. The size of the pillar can be less than 7 μm. The size of the pillar can be less than 5 μm. The size of the pillar can be less than 2 μm. The size of the pillar can be less than 1 μm. The width of the gap can be varied from 2 μm to a couple of hundred of microns. The width of the gap can be less than 200 μm. The width of the gap can be less than 100 μm. The width of the gap can be less than 50 μm. The width of the gap can be less than 20 μm. The width of the gap can be less than 10 μm. The width of the gap can be less than 7 μm. The width of the gap can be less than 5 μm.

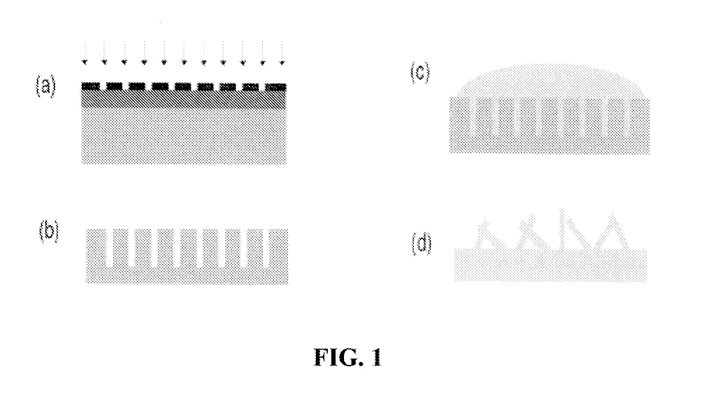

FIG. 1 is a schematic showing the fabrication process of a 3D polymer microstructure. In FIG. 1(a), a silicon mold was etched by standard UV lithography process. FIG. 1(b) is a schematic showing a silicon mold. When treated with silane, the surface of the mold becomes hydrophobic. In FIG. 1(c), polydimethylsiloxane (PDMS) polymer was loaded onto the mold. After polymer was loaded, the setup can be put into a vacuum chamber for degassing. After curing, the PDMS layer can be released from the mold. In FIG. 1(d), right after releasing, the PDMS microstructures can collapse and touch each other due to the interfacial/capillary force. In FIG. 1, black color indicates chrome mask; grey color indicates silicon wafer; and blue color indicates PDMS.

Self-assembled 3D micro structures can be created with interfacial force or capillary force. During the process, no complicated multilayer mask lithography or chemical etching process needs to be involved. The releasing of the polymer can also be achieved in an organic solvent, such as ethanol. Afterwards, the polymer can be left in the air or on the hotplate to dry to utilize capillary force to cause the collapse of the PDMS microstructures and form 3D structures.

In addition to PDMS microstructures, other polymer microstructures can be prepared, such as polystyrene, PMMA, epoxy resin, poly(ethylene glycol) (PEG), poly(ethylene glycol) diacrylates, dimethacrylates, tetraacrylates, a copolymer thereof, and so on. Epoxy resin can include 1002F EPON.

The mold material can be silicon, metal, polymer, ceramic, or glassy structures. Mold can be fabricated by using lithography, using laser etching, or similar methods. Lithography includes soft lithography, nanolithography, photolithography, UV lithography, electron beam lithography, maskless lithography, nanoimprint lithography, interference lithography, or X-ray lithography. Laser etching is a process that deposits or processing material using laser. Laser etching can include laser direct write.

### Using 3D Microstructure for Cell Culture

Cells in the body respond dynamically to their microenvironment. The composition and physical properties of the extracellular environment can modulate both cellular fate and function. For example, the controlled interaction of cells with specific extracellular architectures is critical to maintaining cell phenotype. Cells can respond to changes in mechanical cues such as gel elasticity, porosity and density. A much sought after goal in tissue engineering is the fabrication of 3D tissue scaffolds that incorporate both microscale chemical and mechanical domains.

Microstructures for cell culture can be prepared by lithography. For example, preparing microstructures for cell culture applications can include fabricating highly porous, thin-walled structures, creating cell adhesion patterns on strongly curved surfaces by using thermoforming and a mask based UV irradiation, and integrating nanotopographies by a nanoimprint process. PEG-based hydrogel microstructures can be fabricated using photolithography to encapsulate viable mammalian cells.

In another method, a microwell array can be used for 3D cell culture. The fabricated microwell array can have 3D microstructures within each well. The three-dimensional microstructure can include collapsed polymer columns, wherein a partial surface of each collapsed polymer column contacts with a partial surface of at least another collapsed polymer column Each collapsed polymer column can have substantially similar height and size dimensions if straightened. Alternatively, at least two collapsed polymer columns can have substantially different height and size dimensions if straightened.

Three-dimensional microstructure can comprise a plurality of collapsed polymer columns, wherein a portion of a surface of each collapsed polymer column contacts with a portion of a surface of at least another collapsed polymer column; and the surface of each collapsed column includes nanoscale grooves. Nano grooves can be formed during the etching process of the holes on the mold. When liquid PDMS was poured onto the mold and cured on hotplate. PDMS can duplicate these nano grooves structures onto the final pillars. The mold is a reversed version of the PDMS pillar. PDMS can duplicate the structures on the mold with reversed pattern. For example, the holes on the mold will be pillar on the final PDMS device. This fabrication process can be “Soft Lithography”. Each collapsed polymer column can have substantially similar height and size dimensions if straightened. Alternatively, at least two collapsed polymer columns can have substantially different height and size dimensions if straightened.

A method of culturing cells can include adding cells to a microwell including a three-dimensional microstructure and incubating the cells in the three-dimensional microstructure. A different method of culturing cells can include adding cells to a surface including a three-dimensional microstructure. The three-dimensional microstructure comprising a plurality of collapsed polymer columns, wherein a portion of a surface of each collapsed polymer column contacts with a portion of a surface of at least another collapsed polymer column, and the surface of each collapsed column includes nanoscale grooves. Each collapsed polymer column can have substantially similar height and size dimensions if straightened. Alternatively, at least two collapsed polymer columns can have substantially different height and size dimensions if straightened.

Self-assembled complex PDMS 3D microstructures can be fabricated utilizing the instability of HAR micropillars without the need of fabricating complicated replica mold. Such 3D microstructures can be controllable by varying the parameters such as the micropillar size, length, gap, etc. Moreover, by simply tuning the DRIE process when fabricating mold, the micro/nano hybrid 3D structures can be obtained. The method offers a great deal of flexibility in modifying extracellular microenviroment which is of great significance for in-vitro cell study. Furthermore, the culture of cell lines, such as SaOs-2 and NIH-3T3 cell lines, on the micro-forest chip can provide strong interactions between the cells and the PDMS 3D microstructures, indicating the potential of applying the technique to simple, inexpensive and efficient 3D cell research microdevice.

### Nanoparticles Polymer Composite

The mixing of polymers and nanoparticles is opening pathways for engineering flexible composites that exhibit advantageous electrical, optical, mechanical, or optical properties. Directing spatial distribution of nanoparticles can control the macroscopic performance of the composite.

Magnetic nanoparticles are a class of nanoparticle that can be manipulated using magnetic field. Sizes of nanoparticles are generally smaller than 1 micrometer in diameter. A magnetic nanoparticle, or a ferromagnetic nanoparticle, can include magnetic elements such as iron, nickel, cobalt, their chemical compounds, or their alloy, or the like. Additionally, the magnetic nanoparticles may be bimetallic or trimetallic, or a mixture thereof. Examples of suitable bimetallic magnetic nanoparticles include, without limitation, CoPt, FePt, FeCo, MnAl, MnBi, CoO.Fe₂O₃, BaO.6Fe₂O₃, mixtures thereof, and the like. Examples of trimetallic nanoparticles can include, without limitation, tri-mixtures of the above magnetic nanoparticles, or core/shell structures that form trimetallic nanoparticles such as Co-covered FePt.

The magnetic nanoparticles may be prepared by any method known in the art, including ball-milling attrition of larger particles, followed by annealing. The nanoparticles can be made directly by radio frequency plasma. The nanoparticles can also be made by a number of in situ methods in solvents, including water.

Magnetic nanostructures offer important advantages in comparison with non-magnetic nanoparticles. Firstly, due to their magnetic nature, they can be activated, controlled and manipulated remotely with a magnetic field. Additionally, their size can be controlled within a range extending from a few nanometers in size to hundreds of nanometers in size, putting them in the same size range as many biological entities like enzymes, cell receptors, genes, bacteria, etc. Magnetic nanoparticles possess attractive properties which can see potential use in catalysis including nanomaterial-based catalysts, biomedicine, magnetic resonance imaging, magnetic particle imaging, data storage, environmental remediation, nanofluids, and optical filters.

Magnetic actuated microdevices can be used to achieve several complex functions in microfluidics and microfabricated devices. For example, magnetic mixers and magnetic actuators can help handling fluids at a small scale. Magnetically actuated micropillar arrays can be created. Dispersion of magnetic nanoparticles within polymeric micropillars can help design micrometre scale magnetic features. By creating a magnetic field in the vicinity of the substrate, well-defined forces can be applied on these magnetic nanoparticles which in turn induced a deformation of the micropillars. By dispersing magnetic nanoparticles into the polymer micropillars, synchronized motions of a group of pillars or the movement of isolated pillars can be induced under a magnetic field. When combined with microfabrication processes, this versatile tool leads to local as well as global substrate actuations within a range of dimensions that are relevant for microfluidics and biological applications.

A three dimensional microstructure can include a plurality of polymer pillars on the surface of a substrate, wherein each polymer pillar includes a plurality of nanoparticles. The nanoparticles can include magnetic material, such as iron, cobalt, nickel, or a compound thereof, or an alloy thereof. When the nanoparticles include iron material, the weight percentage of iron can be more than 90% of the polymer pillars; the weight percentage of iron can be more than 70% of the polymer pillars; the weight percentage of iron can be more than 50% of the polymer pillars. Pillar size is the diameter of a pillar. For a square shape pillar, pillar size refers to the length of each side of the cross section of the pillar. The size of a polymer pillar can be less than 20 μm; the size of a polymer pillar can be less than 10 μm; the size of a polymer pillar can be less than 5 μm. The height of a polymer pillar can be less than 100 μm; the height of a polymer pillar can be less than 50 μm; the height of a polymer pillar can be less than 20 μm.

Nanoparticles can be grown on a patterned three-dimensional (3D) polymer microstructure. In one method, the technique can integrate 3D direct writing of heterogeneous microstructures with nanoparticle synthesis. Metal nanoparticles can be attached to a patterned polymer microstructure across three dimensions by combining multi-material digital photopolymerization with nanoparticle synthesis involving a special reducing agent.

Other methods preparing composite polymer microstructures include directly mixing the nanoparticles into the polymer before loading onto the mold or first suspending nanoparticles into solvent and then replace the solvent with polymer and then load the polymer onto the mold. These methods have the issue of either nanoparticle aggregation, which prevents fabricating small microstructures, such as less than 5 μm, or involving complicated chemical process to treat the surface of the nanoparticles preventing the aggregation and to replace the solvent with polymer. Aggregation of magnetic particles limits the application of solvent-casting techniques to fabricate magnetic micropillar structures with highly viscous polymers, such as polydimethylsiloxane (PDMS).

Another method for preparing a nanoparticle polymer composite can include loading an organic solvent containing magnetic nanoparticles onto a mold having a three-dimensional pattern including a plurality of columnar features, applying a magnetic field to the mold to move the nanoparticles into the three-dimensional pattern of the mold, removing at least 70% of the organic solvent from the three-dimensional pattern of mold; loading a polymer onto the mold, and forming the three dimensional microstructure by releasing the polymer from the mold, wherein the three dimensional microstructure includes polymer pillars containing magnetic nanoparticles. This method simplifies fabrication process, improves fabrication efficiency and reduces the cost of fabrication.

### Device Using Polymer Nanoparticle Composite

A plurality of polymer pillars, wherein each polymer pillar includes magnetic nanoparticles, can be used in a device. When including a magnetic field source that can deform the polymer pillars, the device can be used in many applications, such as studying the movement of a subject (for example, a cell or a nematode), as a microfluidic micromixer, and etc.

Plant nematodes are major pathogens of plants, interacting with other pathogenic micro-organisms in disease complexes, and in some instances constituting the main cause of damage to plants. Resulting losses are experienced as reduced yields, downgrading and unmarketability of produce, and restrictions on local and international trade in plants and plant products. Global crop losses due to nematode parasitism are in the order of billions of dollars. It can be important to study the movement of nematode.

A nematode study device can include a plurality of polymer pillars, wherein each polymer pillar includes magnetic nanoparticles and a magnetic field source that deforms the polymer pillars when operating. A magnetic field source is a source that can generate a magnetic force or a magnetic field. For example, a magnetic field source can be a magnet or a electromagnet. A magnetic source can also come from moving charges.

Once nematode is loaded onto the Fe nanoparticles filled pillar array, a magnet can be added externally to attract the pillars move toward the magnet position. The magnet will be moved into different position in a constant or varied frequency. The pillars can deform with the move of the magnet and thus provide a mechanical stimulation to the nematode on the pillar array. The study of the response of the nematode under such mechanical stimulation can provide more insight to the biological information of nematode.

Polymeric micropillar devices can also be used for cell studies. Cell movement or motility or migration is a highly dynamic phenomenon that is essential to a variety of biological processes. Cells can reach their target by crawling. Cell migration is a central process in the development and maintenance of multicellular organisms. Tissue formation during embryonic development, wound healing and immune responses all require the orchestrated movement of cells in particular directions to specific locations. Errors during this process can have serious consequences, including mental retardation, vascular disease, tumor formation and metastasis. An understanding of the mechanism by which cells migrate may lead to the development of novel therapeutic strategies for controlling, for example, invasive tumor cells.

Cells in the body respond dynamically to their microenvironment. Cells often migrate in response to specific external signals, including chemical signals and mechanical signals. The composition and physical properties of the extracellular environment can modulate both cellular fate and function. For example, the controlled interaction of cells with specific extracellular architectures is critical to maintaining cell phenotype. Cells can respond to changes in mechanical cues such as gel elasticity, porosity and density.

Device using polymer micropillars containing magnetic nanoparticles can be used to study cell mechanics. Cells can be loaded onto these devices. When cells begin to adhere to the top surface of pillars, cells can exert traction force to the pillars underneath them and this will lead to the deformation of the pillars. If a magnetic source is applied, the deformation of the pillar can be limited or even overcome by the magnetic force. With different field strength magnet, the degree of overcome of the pillar deformation will be different. Such overcome force can be sensed by the cells and cells may modify their morphology to adapt to such overcome force. Such modification of the cell morphology may lead to complicated biological change within cells and so will affect cell adhesion, signaling and proliferation. Thus, the Fe nanoparticles filled PDMS pillar array can be used as the cell mechanics study device.

Microfluidics describes flow in devices having dimensions ranging from millimetres to micrometres and capable of handling volumes of fluid in the range of nano- to microliters. Microfluidics technology can be used in ink-jet printing, lab-on-a-chip assays, reagent delivery, drug development, food and chemical industries, microelectrical mechanical systems, biomedical device and nanotechnology.

Fluidic mixing is a basic step for many analytical applications and becomes more challenging with miniaturization of the fluidic system. The aim of microfluidic mixing is to achieve a thorough and rapid mixing of multiple samples in microscale devices. A mixer can be a critical component in microfluidic devices. For example, mixing of reaction components is essential for providing homogeneous reaction environments for chemical and biological reactions. The efficiency of many devices depends on mixing. In other applications, rapid and controlled mixing is essential for studying reaction kinetics with much better time resolution as compared to microscale techniques.

Methods of microfluidic mixing can include electroosmotic mixing by periodically varying the electric field to mix two aqueous flows, time pulsing by pulsing the flow rate in one of the inlets, ultrasonic mixing using a piezoelectrically driven diaphragm at ultrasonic frequencies, bubble mixing by interaction of the fluid with bubbles of gas introduced into the channels, or magnetic stirring by using a magnetic field to rotate one or more magnetic bars within the fluid medium.

Magnetic stirring can be caused by polymer pillars comprising magnetic nanoparticles, such as Fe nanoparticles. Polymer pillars comprising magnetic nanoparticles can also be used to mixed different kinds of liquids. For example, when two different liquids were injected into a microfluidic channel, the nanoparticles filled pillar can swing or swirl with the external magnetic force to mix the liquid.

### Examples

### Materials and Methods

Materials.

Dulbecco's modified Eagle medium (DMEM), fetal bovine serum (FBS), Trypsin, Alexafluo 488 phalloidin and penicillin/streptomycin (P/S) were ordered from Life Technologies (Carlsbad, Calif., USA). Human plasma fibronectin, Vinculin Monoclonal Antibody, Fluorescent-labeled anti-mouse secondary antibody and DAPI were obtained from Millipore (Billerica, Mass., USA). All other reagents were purchased either from VWR (Leicestershire, UK) or Fisher Scientific (Loughborough, UK). Human osteosarcoma cell line SaOs-2 cells and mouse fibroblasts NIH-3T3 cells were ordered from ATCC (Manassas, Va., USA).

SEM observation used scanning electron microscopy (SEM, Quanta 200, FEI Inc., USA). Prior to the observation, the sample was sputter coated with the gold at depth of 7 nm.

PDMS 3D Microstructure Fabrication.

The device was fabricated by one step casting replication process, FIG. 15(A). A silicon wafer mold with an array of microholes (the size of the pattern region is 1×1 cm) were fabricated by standard photolithography and DRIE etching and then was salinized with (tridecafluoro-1,1,2,2-tetrahydrooctyl)-1-trichlorosilane for overnight. See, for example, Xia, Y. N. et al., Angew Chem Int Edit 37, 551-575 (1998); Tan, J. L. et al., P Natl Acad Sci USA 100, 1484-1489 (2003), each of which is incorporated by reference in its entirety. The mixture of PDMS base and curing reagent at ratio 10:1 was poured over the salinized silicon mold and cured at 100° C. for 10 mins. The cured PDMS layer was peeled off in the air and, during the peeling process, the adhesive interactions of neighboring micropillars and substrate cause the collapse of micropillars which results in self-assembled 3D microstructures on the PDMS substrate. Before the usage, the device was treated in a plasma cleaner and soaked into pure EtOH under vacuum for degasing. Then the pure EtOH was exchanged into 70% EtOH for sterilization. After 5 mins soaking in 70% EtOH, the device was taken out inside cell culture hood and washed with phosphate buffer saline (PBS) buffer for 4 times. Before cells were loaded onto the PDMS 3D device, the device was coated with fibronectin (FN) (25 μg/mil, 2 hr) and washed 3 times by PBS buffer.

3D Microstructure Characterization.

To characterize the microstructures, some parameters are introduced: the substrate coverage ratio A that defines area ratio of the regions covered by the collapsed micropillars to the whole 3D microstructure region in the view of SEM image. Those uncovered regions were identified as the regions where PDMS substrate can be observed in the top view SEM micrographs. The second factor (N) is the maximum number of overlaid collapsed micropillars in vertical direction and describes the complexity of the 3D microstructure in Z direction. The third factor (H) is the average 3D microstructure layer height. All these factors were obtained by manually measuring within top view and/or side view of SEM micrographs at the magnification of 4000×. The area ratio was measured by ImageJ.

Cell Culture and Imaging.

NIH/3T3 cells were cultured in DMEM medium and SaOs-2 cells were cultured in a mixture medium of DMEM and F-12K at the ratio of 1:1. Both medium were added with 10% FBS and 5% P/S. Both cell lines were maintained in a standard incubator with humidity of 95% and 5% CO2. Imaging was performed on an upright Zeiss LSM710 confocal microscope equipped with 40× and 63× water immersion objective lens.

Cell Fixation and Staining.

For confocal imaging, cells on the device were rinsed once with 1×PBS and then fixed with 4% paraformaldehyde (PMA) for 20 min. The cells were then permeabilized with 0.1% Triton X-100 for 5 min at room temperature. After that, the cells were washed with wash buffer and applied with 1% bovine serum albumin (BSA) blocking solution for 30 min at room temperature. Then cells focal adhesions were stained with primary antibody (Anti-Vinculin, 1:200 dilution), the TRITC-conjugated secondary antibody (IgG 1:200 dilution) and Alexa Fluor 488 phalloidin, following the manufacture's instructions.

For SEM observation, cells on the device was rinsed once with 1×PBS and then fixed Glutardehyde (2.5% in Cacodylate Buffer) for 1 h at room temperature. The fixed cells were then washed by 1×PBS for one time. Then the cells were soaked by the ethanol for 5 mins at the increasing concentration with the starting concentration of 20% and the step concentration of 10% until the final concentration 100%. After that, the cells on the device were taken out and left in the air to dry at room temperature.

### Collapse of HAR PDMS Micropillars

FIG. 15 describes a fabrication process flow of the PDMS 3D microstructures (A) and SEM image of the top view (B) and side view (C) of the fabricated PDMS 3D microstructures, micropillar size 5 μm. The arrows in FIG. 15(C) are pointing three types of collapsed micropillars: tilted micropillar (white arrow), bowed micropillar (red arrow) and curved micropillar (green arrow). Scale bar is 10 μm.

The microforest in FIG. 15(B), formed by the collapse of high aspect ratio (HAR) micropillars during PDMS peeling process in the air, was the result of the combination of two different types of collapses: a) the ground collapse, and b) the lateral collapse, which are caused by adhesive interactions between micropillars and the substrate and the adhesion between neighbor micropillars respectively. See, for example, Chandra, D. et al., Accounts of Chemical Research 43, 1080-1091 (2010); Roca-Cusachs, P. et al., Langmuir 21, 5542-5548 (2005), each of which is incorporated by reference in its entirety.

The critical ground collapse aspect ratio, (h/d)_{c}, of the micropillar, has been derived by Roca-Cusachs et al. as:

$${( \frac{h}{d} )}_{c} = \frac{{π}^{5 / 3}}{{2}^{11 / 33 \; 1 / 2}} {( 1 - {ϑ}^{2} )}^{- \frac{1}{6}} {( \frac{E}{2 {γ}_{SV}} )}^{2 / 3} {d}^{2 / 3} \qquad ( 1 )$$

Where h is the length of the micropillar, d is the diameter of the micropillar, θ is the Possion ratio, E is theYoung's modulus of the PDMS and γ_{SV} is the surface energy of the PDMS pillar. The critical aspect ratio for square shape micropillar lateral collapse has been derived by Glassmaker et al. as:

$${( \frac{h}{d} )}_{c} - {( \frac{3 {Ew}^{2}}{S d {γ}_{SV}} )}^{1 / 4} \qquad ( 2 )$$

Where w is the gap between adjacent micropillars. See, for example, Glassmaker, N. J. et al., Journal of the Royal Society Interface 1, 23-33 (2004), which is incorporated by reference in its entirety.
The aspect ratio of the PDMS micropillars can exceed the critical aspect ratio. Due to the combination of two collapse modes, unlike discrete cluster array formed by the micropillar collapse induced by the capillary force, the HAR PDMS micropillars on the device collapse in the air and overlapped on each other so that they formed a continuous and complex 3D microstructures, FIG. 15(B). A detailed observation of the 3D microstructure side view further revealed that the collapsed micropillars contained three patterns of the collapse: tilted, bowed (collapsed micropillar with one turn) and curved (collapsed micropillar with two or more turns) micropillars, FIG. 15(C). These three types of collapsed micropillars actually represented the different degree of deformation of an individual pillar during the formation of the microforest, the curved micropillars were the ones with highest degree of deformation. The mixture of these three type collapsed micropillars formed the final microforest structures.

### Preparation of Polydimethylsiloxane (PDMS) Microstructure

PDMS microstructure can be prepared by etching a silicon mold by standard UV lithography process, treating the silicon mold with silane to make surface hydrophobic, loading PDMS polymer onto the mold and degassing the mold and the polymer in a vacuum chamber, curing PDMS, and releasing the PDMS layer from the mold. After releasing, the PDMS microstructures collapse and touch each other due to the interfacial/capillary force.

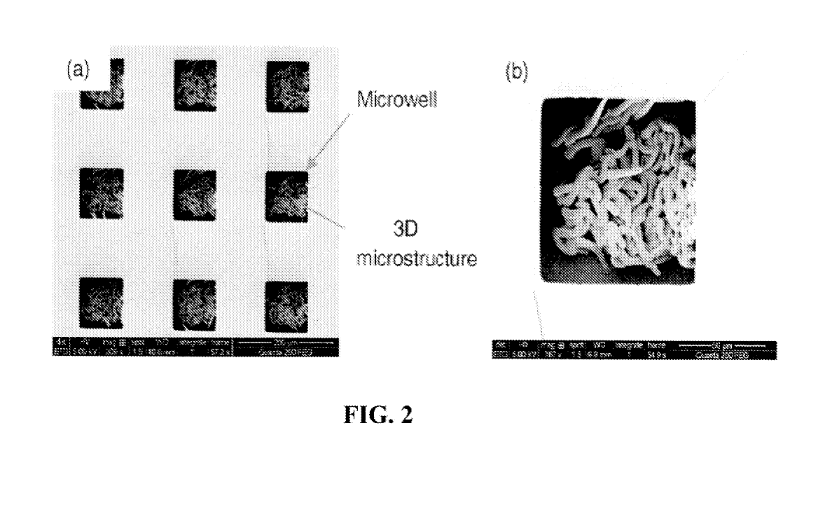

PDMS microstructure can be fabricated in a microwell array. FIG. 2(a) shows an SEM image of a microwell array with 3D microstructures in each well, and FIG. 2(b) shows details of the PDMS microstructure inside a single microwell. These SEM images show that the 3D microstructures were formed by collapsed PDMS micropillary array within a microwell array. The PDMS pillar size in FIG. 2 is 2 μm. In a different embodiment, PDMS microstructure can be fabricated in a surface.

Different PDMS Microstructures Formed with Different Molds

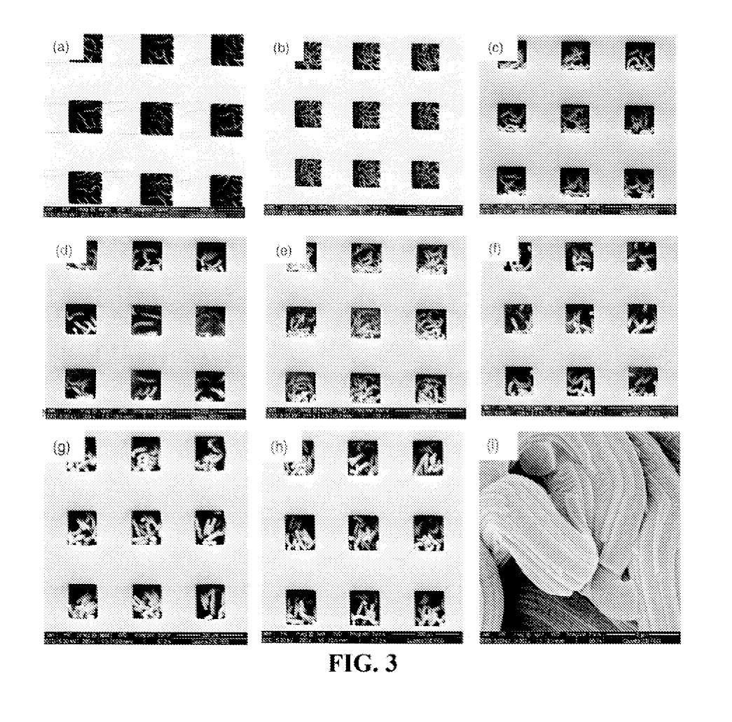

Pillar size, height, gap and shape can be varied to form different 3D structures. FIG. 3 shows SEM images of microwell arrays with various PDMS 3D microstructures, which were formed by molds with different size and gap pillars. The designs include 2 μm size 10 μm gap in FIG. 3(a), 2 μm size 5 μm gap in FIG. 3(b), 5 μm size 7 μm gap in FIG. 3(c), 5 μm size 10 μm gap in FIG. 3(d), 5 μm size 5 μm gap in FIG. 3(e), 7 μm size 10 μm gap in FIG. 3(f), 7 μm size 7 μm gap in FIG. 3(g), and 7 μm size 5 μm gap in FIG. 3(h). FIG. 3(i) shows a detailed image of 2 μm size and 5 μm gap pillar formed 3D microstructure inside a single microwell. Nano groove patterns can be formed on the side wall of the pillar via tuning the deep reactive ion etching (RIE) silicon mold etching process. A pillar in the 3D microstructure is a polymer column. For a 3D microstructure, pillar size is the diameter of a polymer pillar, such as a PDMS pillar. If a pillar is square shape, pillar size refers to the length of each side of the cross section of the pillar. A gap in the 3D polymer microstructure is the space between polymer pillars. When referring to a 3D polymer microstructure, the width of a gap is the distance between the edges of two adjacent pillars before the polymer columns collapse.

PDMS 3D Microenvironments with Different Geometric Parameters

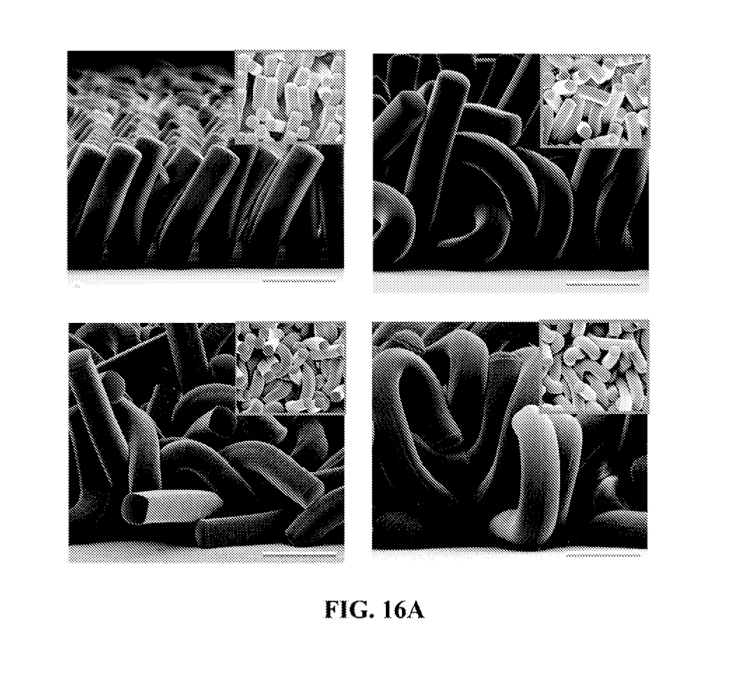

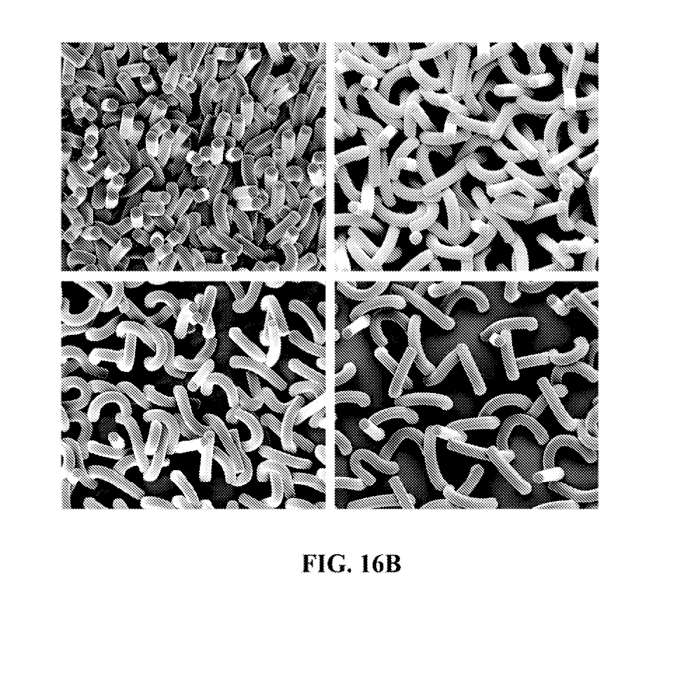

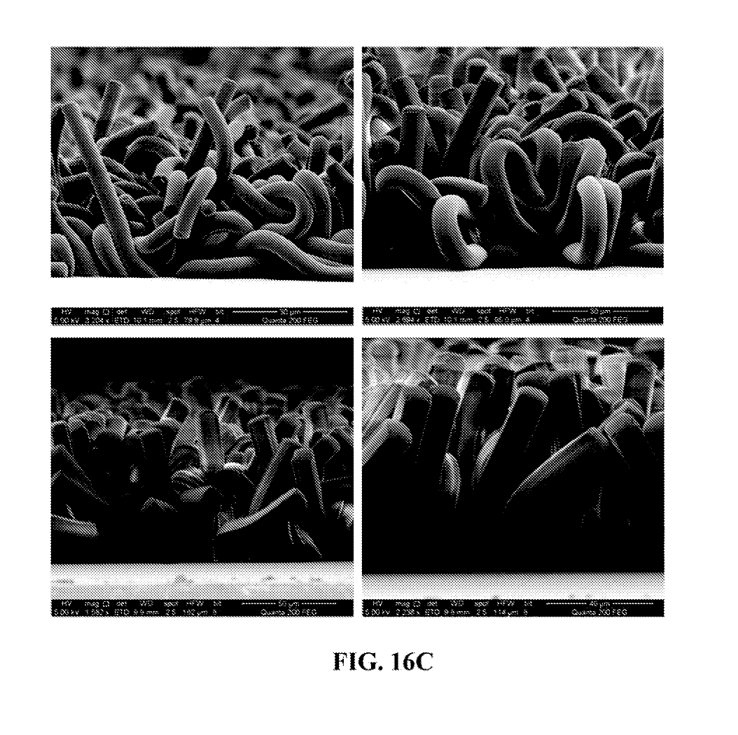

To investigate the influence of geometric parameters of the square shape micropillars on to the formation of PDMS 3D extracellular microenvironements, a series of micropillars arrays with various length (1), width (a) and edge to edge spacing (s) were fabricated. The top view and the side view of the 3D microstructures were imaged with scanning electron microscope (SEM), FIG. 16.

On the 3D microstructures devices, gap regions between collapsed micropillars were inter-connected. This showed that micropillar dimensions did not affect the 3D microstructure inter-connectivity. The inter-connectivity can be critical for cell culture on 3D microstructures. See, for example, Sun, J. X. et al., J Mater Sci-Mater M 22, 2565-2571 (2011), which is incorporated by reference in its entirety. But dimensions affected the percentage of the collapsed micropillars adhering to the substrate and the percentage of the curved micropillars among all collapsed micropillars on a single device. For example, on the 3D microstructures formed by micropillars (a=5 μm, 1=55 μm) with various spacing, it was found that only 1-2% of the micropillars adhered to the substrate when micropillar spacing was 5 μm. On the device with 20 μm micropillar, the less micropillars were found adhered to the substrate and the more curved micropillars appeared, the more complex final 3D microstructures resulted.

It is because the ground collapse became dominated at larger gaps. Therefore, with the increase of the gap, the formed 3D microstructure was composed mainly of bowed micropillars rather than curved ones, which results a 3D microstructure with less complexity. These observed differences can be attributed to the rigidity of the micropillars. Indeed, the spring constants of the PDMS micropillars, which represented the stiffness of the micropillars, increased with the size of the micropillar and decreased with the length of the micropillar. The relationship, as described by Ghibaudo et al., can be summarized by the following formula:

$${k}_{n} ∝ E \cdot \frac{{d}^{2}}{L} and {k}_{t} ∝ E \cdot \frac{{d}^{4}}{{L}^{3}} \qquad ( 3 )$$

Where k is the PDMS micropillar spring constant under compression (kₙ) and under shear (kₜ). E is the PDMS Young's modulus, d is the diameter of the micropillar and L is length of the micropillar. See, for example, Ghibaudo, M. et al., Lab on a Chip 11, 805-812 (2011), which is incorporated by reference in its entirety. The smaller the size, the softer the micropillar is. Therefore, 3 μm micropillars can form curved shape deformation when collapsed and bended more to the substrate due to its low stiffness, which lead to the smallest average height. The 10 μm micropillars, on the contrary, were mainly tilted or bowed, which lead to less complexity in z direction and the highest average 3D microstructures height.

For micropillars of different sizes (g=5 μm and 1=55 μm), the substrate coverage ratio is nearly 100%. In Z direction, 3 μm and 5 μm micropillar chips had 3˜4 overlaid micropillars and some 3 μm micropillar chips even have 5 overlaid micropillars. On 7 μm and 10 μm micropillar chips, there were only maximum 2 overlaid micropillars. In addition, on 10 μm micropillar chips, more than half collapsed micropillars were tilted micropillars (≈65%), bowed micropillars were also found (≈35%) but no curved micropillars was found. The average 3D microstructures height increased with the increase of the micropillar size from 31±3 μm at 3 μm micropillar chip to 48±2 μm at 10 μm micropillar chips, see FIG. 16(C).

Such spring constant relationship was also observed in 3D microstructures chips with 5 μm size, 5 μm gap and various length micropillars. Shorter micropillar had lower spring constant and was stiffer than the higher micropillars, so they were harder to be deformed. Therefore, on 35 μm long micropillar chip, only tilted and bowed micropillar were observed and as a result N=2, but, on 55 μm long micropillar chip, not only tilted, bowed but also curved micropillars were found and so N=4 (see FIG. 16(A)). For the same reason, the average 3D microstructures height thickness decreased (H) only increased about 10 μm (from 29±2 μm to 39±4 μm) when micropillar length increased 20 μm (from 35 μm to 55 μm). Meanwhile, substrate coverageratio (A) increased from ≈85% to 100%.

Based on the above results and analysis, the 3D microstructures fabricated using the PDMS micropillar array can be controlled by modifying geometric parameters of the micropillars to satisfy the different requirements of 3D cell culture study. The mixture of the different size of micropillars on the same chip is also doable to further tune the final 3D microstructures. More importantly, such a tuning will not increase the complexity of the fabrication.

### Hybrid PDMS 3D Microenvironment Fabrication

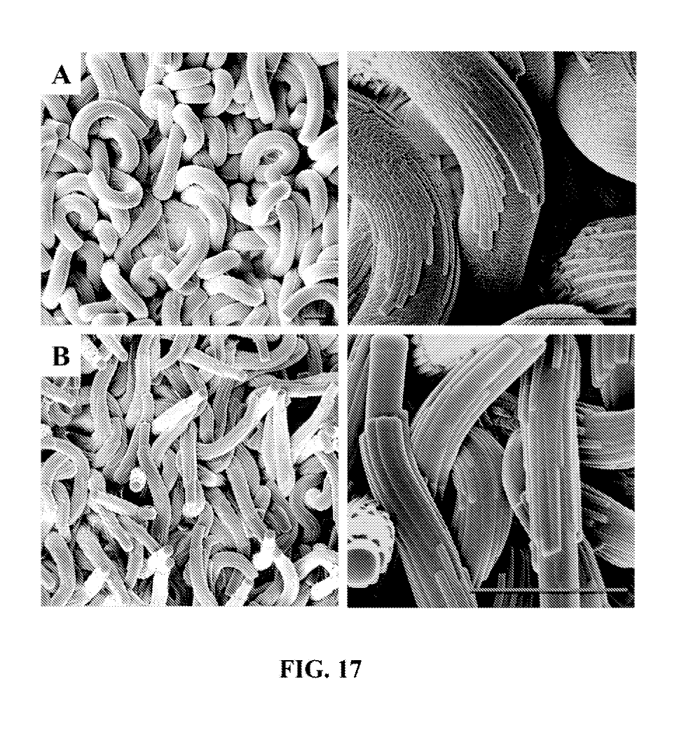

Nanostructure can have a significant influence on cell behaviors. See, for example, Schafer, U. et al., Journal of Tissue Engineering and Regenerative Medicine 6, 83-84 (2012); Prabhakaran, M. P. et al., Biomedical Applications of Polymeric Nanofibers 246, 21-62 (2012); Schlatt, S. et al., Biology of Reproduction 85 (2011); Il Cho, Y. et al., Acta Biomater 6, 4725-4733 (2010); Sjostrom, T. et al., Acta Biomater 5, 1433-1441 (2009); Yim, E. K. F. et al., Experimental Cell Research 313, 1820-1829 (2007); Chang, Y. C., Abstr Pap Am Chem S 232 (2006); Muth, C. A. et al., Plos One 8 (2013), each of which is incorporated by reference in its entirety. If the nanostructure can be fabricated onto the 3D microstructures, it will provide more applications of PDMS 3D microstructures in biological cell study. A deep RIE etching recipe can be developed, by which 300 to 500 nm nanoline structures can be created on the side wall of microholes on the silicon mold. Unlike the standard deep RIE process where smooth side walls and lower scallop size are desired and obtained by balancing the deposition and etching rates, an imbalance between the deposition and etching can be created by increasing the etching cycle time (1 s increase). Due to the unbalanced deposition and etching rates, the polymer protection deposited on the side wall of the silicon microholes on the mold is etched completely thereby creating an opportunity to increase the scallop size. This increased scallop size translates to a rough nanoline structure on silicon microhole sidewall. After etching, the residual polymer and photoresist are removed by oxygen plasma, the nanostructured sidewalls of the microholes on the mold were achieved. After soft lithography, the nanoline shaped nanostructures were faithfully replicated to the side wall surface of the micropillars. We named such structures as hybrid 3D microstructures, as shown in FIG. 17. The successful fabrication of the hybrid microstructrures to finely tailor the surface topography is often used in biodevice paper of the 3D microstructure so that more flexibility is offered for modifying extracellular microenvironment for in-vitro cell research.

### Cell-PDMS 3D Microstructures: Cell Morphology

To test the cellular response to the PDMS 3D microstructures, fibroblast cell line (NIH-3T3) and human epithelial-like osteosarcoma cell line (SaOs-2) were cultured for 96 hrs on the 3D device coated with FN. As a control, these two cell lines were also cultured on the unpatterned flat PDMS control surface coated with FN. The cytoskeleton actin filaments, focal adhesion and nucleus of the cells were visualized by the immunofluorescence staining and observed by confocal microscope. By utilizing z-stack function of confocal microscope and with the aid of Imaris image processing software, 3D structure of the cells was constructed to visualize cell morphology, nuclei deformation and cytoskeleton in a three dimensional way. The SEM imaging was also used to directly visualize the cell morphology.

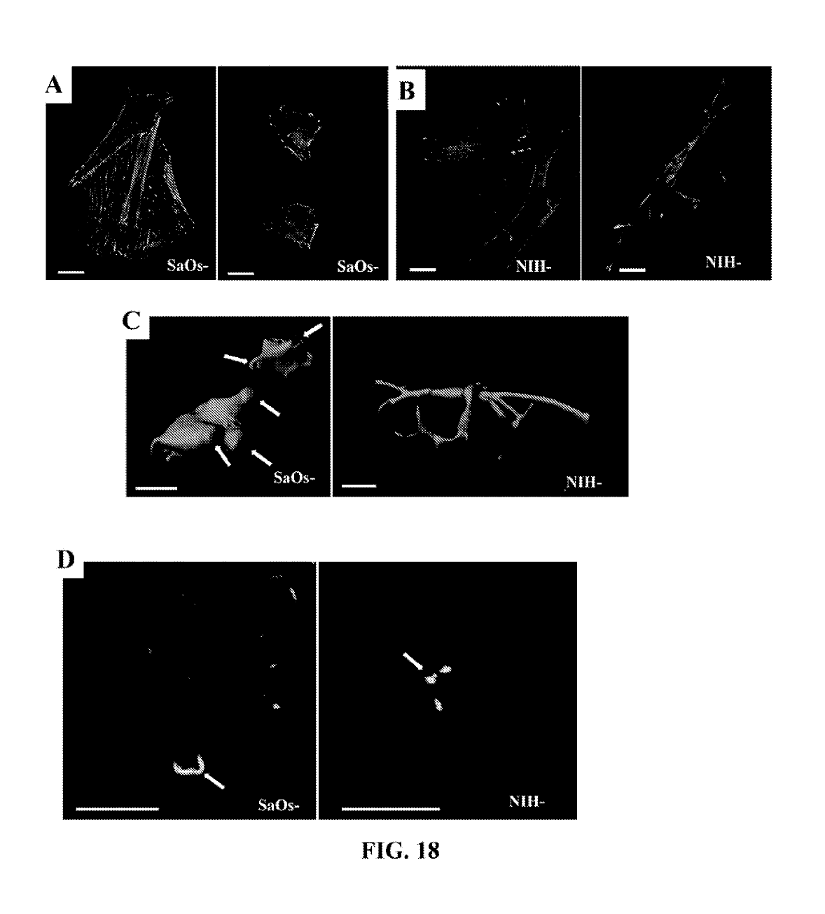

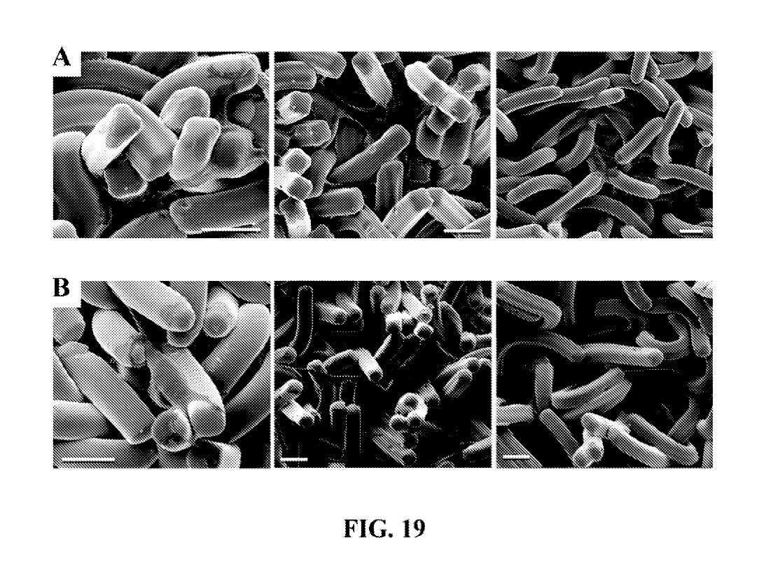

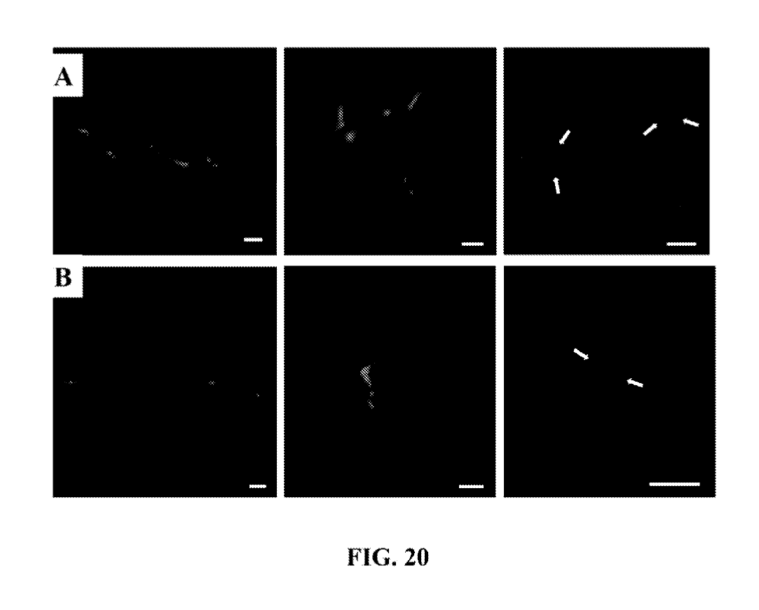

On un-patterned flat PDMS surface, SaOs-2 cells exhibited a spread-out and laterally extended morphology while 3T3 cells showed both laterally extended polygonal morphology and elongated morphology, FIG. 18(A), FIG. 18(B). On 3D microstructure device, both SEM images and confocal 3D renderings revealed that most 3T3 cells demonstrated an elongated and spindle-shaped morphology, while SaOs-2 cells tended to form a compact 3D morphology with projection (confocal maximum intensity projection, MIP) area 40%-60% less than SaOs-2 cells adhered on the flat surface, FIG. 18(A), FIG. 18(B). Moreover, both 3T3 and SaOs-2 cells recognized the geometries of 3D microstructure and accommodate themselves nicely to the 3D microstructures formed by micropillar collapsing, FIG. 18(C) and FIG. 19 (left). The adherence of cells was not just on a single micropillar but on multiple micropillars and the adhesion positions included both the side walls and the top of the micropillars. As a result, cells formed a cell body layer covering multiple micropillars, see FIG. 18(C), FIG. 19 (left).

When the cells met the gap region between micropillars, both types of cells can form either a thick layer of body branch connection or thin filopodia connection to penetrate/across the gap region and created the growth cone on the adjacent micropillar, FIG. 19 (middle). Especially, for NIH-3T3 cells, long spindle-shaped cell bodies can penetrate different gap regions and formed a long connection (can be >100 μm) between different micropillars. In other cases, cells were also observed to directly find their adhesion locations within the gap region, FIG. 19 (middle). On 5 μm gap 3D microstructure device, those cell branch or filopodia connection adhesion locations were found to be the side wall of the micropillars. On 10 μm gap 3D microstructure device, however, due to the large area of flat substrate regions between micropillars exposed to cells, cells were observed to also adhere to the substrate regions. But, even in this case, none of cells was observed to adhere and extend only on the substrate. In contrast, all cells' main body remained adhered to the 3D micropillar and only filopodia or branch connection to the substrate was formed, FIG. 19 (right). Based on the morphology of the cells, both cell lines demonstrated strong reaction to the 3D environment created by the collapsed micropillars.

To further investigate the response of the SaOs-2 cells to the 3D microstructures, actin cytoskeleton and focal adhesion confocal images of cells adhering to the flat PDMS surface and the 3D microstructure device were compared. The confocal MIP images of cells, 3D confocal reconstruction images and the confocal slice images were examined for the comparison.

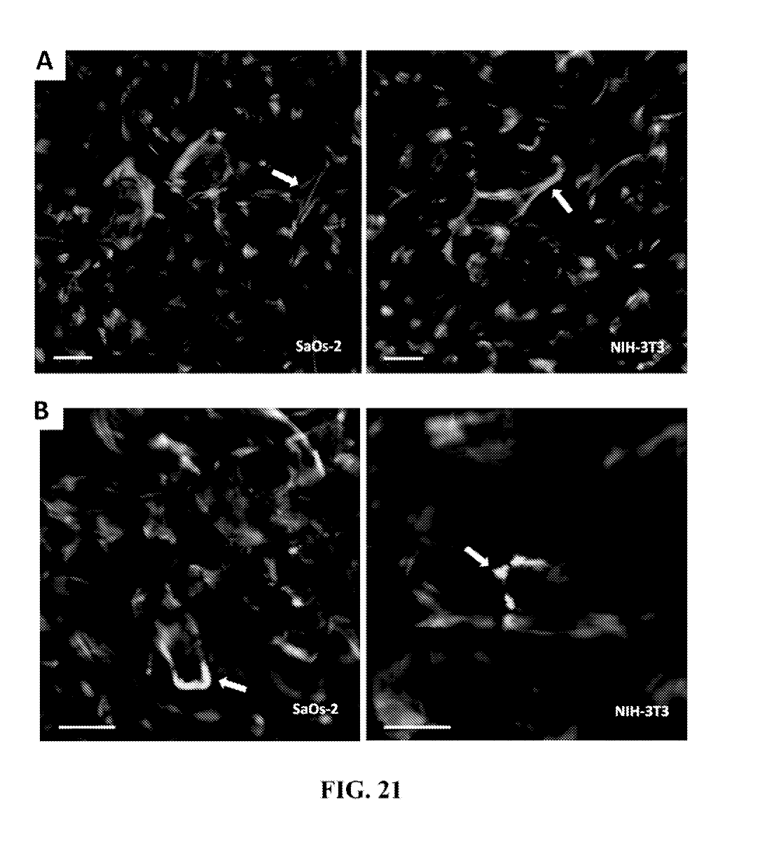

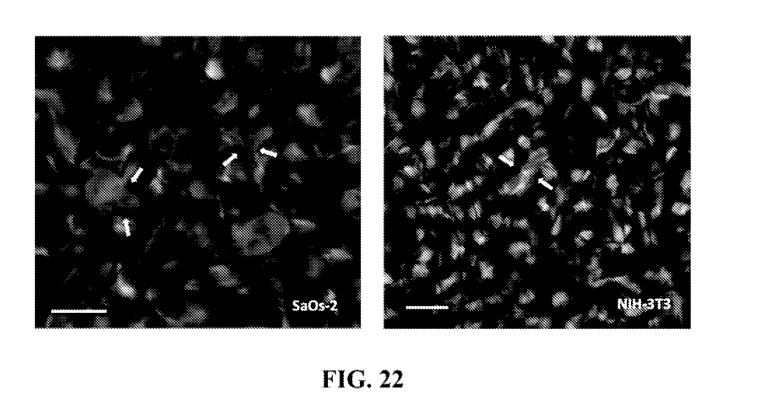

From confocal MIP images, TRITC conjugated Phalloidin stained dense and well-defined F-actin filaments network was clearly observed on the SaOs2 cells and NIH-3T3 cells cultured on unpatterned flat PDMS surface, FIG. 18(A) and FIG. 18(B). On the 3D microstructure device, similar F-actin filaments network was observed, as shown in FIG. 18(A) and FIG. 18(B). No apparent difference in F-actin networks observed in cells cultured on 3D microstructure and on the flat surface can be observed except that the F-actin filaments of both cell lines on the 3D microstructure device were not as clear as the ones on the flat PDMS surface. From the 3D confocal reconstruction images and slice images, it was found that those cellular branches connections between micropillars also contained actin filaments, see FIG. 21(A), which indicated that the 3D geometries did not have major effect on cell actin network formation. In addition, these results agree with those of cells cultured on normal uncollapsed micropillar array. See, for example, Ghibaudo, M. et al., Biophys J 97, 357-368 (2009); Sniadecki, N. J. et al., P Natl Acad Sci USA 104, 14553-14558, doi:DOI 10.1073/pnas.0611613104 (2007); Han, S. J. et al., Biophys J 103, 640-648, doi:DOI 10.1016/j.bpj.2012.07.023 (2012), each of which is incorporated by reference in its entirety.

For focal adhesion (F.A.), it was observed that there were less F.A.s of both cell lines on the 3D microstructures than on the flat surface, FIG. 18(A) and FIG. 18(B). Detailed examination of the confocal slice image of the F.A. of cells on the 3D microstructures revealed that F.A.s localized on the side or top of the micropillars. Some F.A.s were found to have nice alignment to the geometry of side/top of micropillars, FIG. 18(D), which indicated that topographic features can stimulate the recruitment of the vinculin proteins and thus provided another piece of evidence that cells recognized and reacted to the 3D microstructures.

Cell response was also evidenced by the response of the cells' nuclei. First, nuclei of the cells cultured on the 3D microstructures were distributed in 3D. Instead of spreading in a 2D plane as SaOs-2 cells cultured on the flat surface, even for the adjacent cells on 3D microstructures, nuclei of cell colony respond to local environment. Second, about 80% SaOs-2 cells and 40% NIH-3T3 cells were observed to deform their nucleus to either follow the contours of micropillar surface topography where the cell adhered, or adapt to the shape of the gap region, see FIG. 20. It was reported that SaOs-2 cells' nuclei deformed, when loaded onto the normal vertical micropillar array. See, for example, Davidson, P. M. et al., Adv Mater 21, 3586-+(2009); Davidson, P. M. et al., J Mater Sci-Mater M 21, 939-946 (2010); Pan, Z. et al., Biomaterials 33, 1730-1735 (2012), each of which is incorporated by reference in its entirety. The observation indicated that the collapsed micropillar array can also cause severe cell nuclei deformation, which has not been reported before.

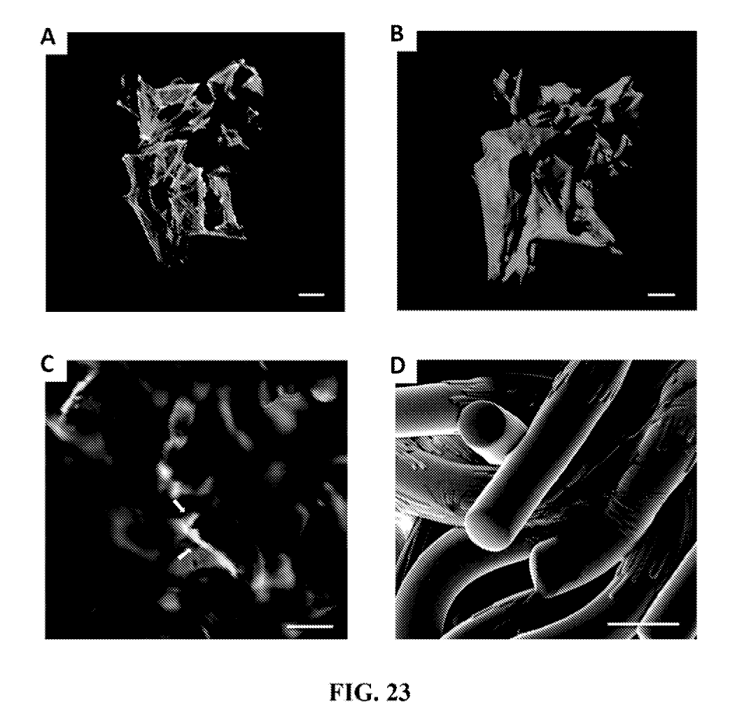

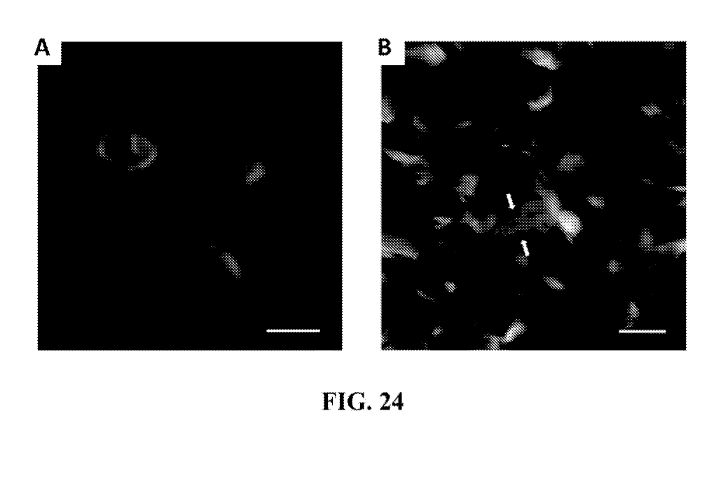

The SEM images, the confocal images of actin filaments, focal adhesion and 3D surface renderings of NIH-3T3 and SaOs-2 cells cultured on the hybrid 3D microstructure were in FIG. 23 and FIG. 24. Similar cell response to the 3D microstructures as described above was observed. Apparent cell response difference between the hybrid 3D microstructure with the normal 3D microstructure has not been observed. Maybe these cell lines do not respond.

### Culturing HeLe Cells on PDMS Microstructures

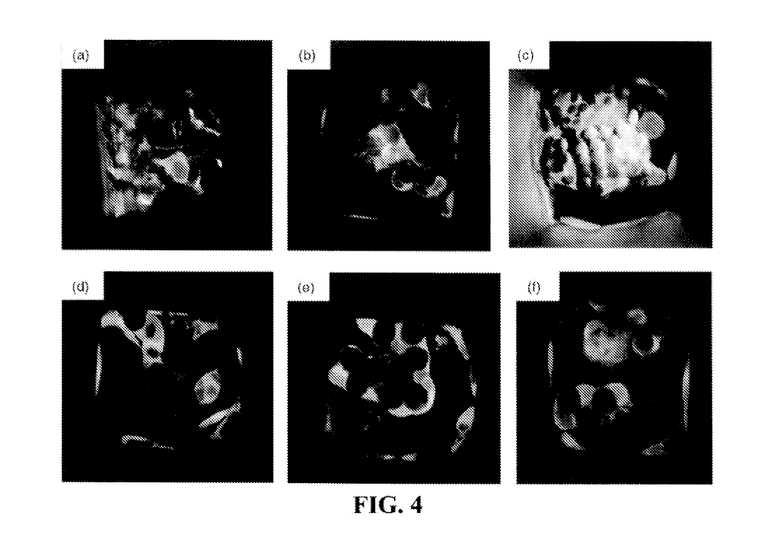

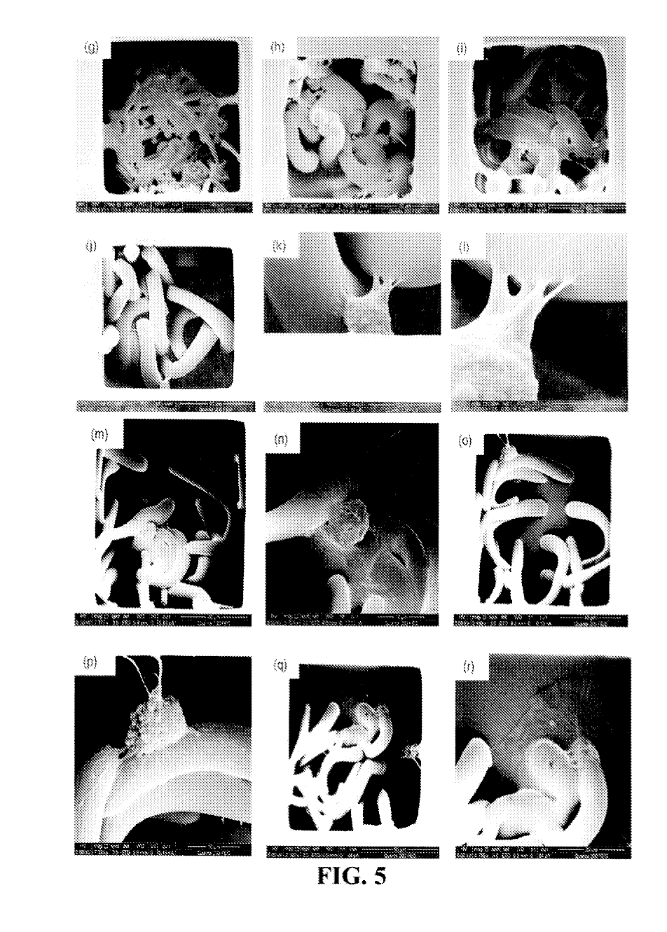

HeLa cells can be cultured on the microwell array that has 3D microstructures for 72 hrs. The cells were stained with live cell dye Calcein Red-Orange and examined under the confocal microscope Zeiss LSM71 G. The confocal images of HeLa cells inside a single microwell were shown in FIG. 4 and FIG. 5. The cells maintained viability within the microwell and grew into the 3D microstructure. The cell colonies show close attachment with the 3D microstructure. In addition, nano-groove structures were created on the side wall of each pillar inside each microwell by tuning the deep RIE etching recipe. Therefore, the cell colonies inside each microwell actually were growing on a nanopatterned 3D microstructure, as shown in FIG. 3(i). This method does not involve complicated fabrication process. Further SEM images (shown in FIG. 4 and FIG. 5) also show that cell colonies appeared in the 3D microstructures.

FIG. 4 shows transmitted light and fluorescence superimposed images in (a), (b), (c), (d), (e) and (f). FIG. 5 shows SEM images of cell colonies on 3D microstructures in a single microwell. FIGS. 5(k), 5(i), 5(n), 5(p) and 5(r) are enlarged view of the cells inside the microwell and attached to the 3D pillar microstructure.

The size and the shape of the pillars can designed with varied values even within a single microwell. For example, there can be 2 μm, 5 μm and 7 μm pillars inside a single microwell to form 3D microstructures. With the varied designs of the pillars, formed 3D microstructures can be various.

### Preparation of Nanoparticle Filled Polymer Micropillars

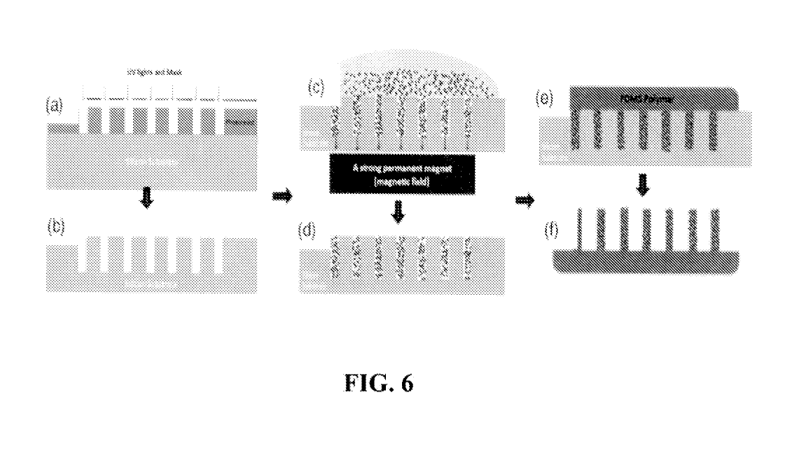

A method of preparing magnetic nanoparticle filled micropillars includes dispersing nanoparticles (for example, Fe nanoparticle) into organic solvent (for example, Acetone), loading the liquid onto the microstructure mold (for example, Silicon mold), then attaching a magnet to the other side of the mold to attract the nanoparticles into the micron range structures on the mold, and loading the liquid polymer (for example, Polydimethylsiloxane, PDMS) onto the mold. After curing, the polymer layer is released from the mold. The replicated microstructures on the released polymer layer will be filled with the nanoparticles. The fabrication process flow of the method is illustrated in the FIG. 6.

After the load of the nanoparticles, the silicon mold was left to dry in the air 10 minutes. This step can be critical for the fabrication since the residue of the organic solvent can prevent the PDMS from filling into the microstructure. After the dry step, PDMS was poured onto the mold. The setup was then put into the vacuum chamber to degas for 45 minutes. Then, the setup was taken out and left on the 100° C. hotplate baking for 30 minutes. Then the PDMS film was peeled off from the mold in ethanol. The PDMS film was dried by using a critical paint dryer.

FIG. 6(a) shows the etching of a silicon mold by standard UV lithography process; FIG. 6(b) shows treating the mold, such as with silane, to make surface hydrophobic; FIG. 6(c) shows loading nanoparticles in organic solvent, such as Fe nanoparticles in acetone, onto the mold with a magnet on the back side of the mold to attract nanoparticles into microstructures; FIG. 6(d) shows drying the mold in air; FIG. 6(e) shows loading polymer, such as PDMS, onto the mold; and FIG. 6(f) shows a three-dimensional microstructure with polymer pillars containing nanoparticles after the polymer was cured and released from the mold. After the polymer was loaded onto the mold, the setup can be put into a vacuum chamber.

This method does not need to suspend the nanoparticles in sticky liquid polymer, which avoids complicated chemical surface treatment of the nanoparticles to keep the nanoparticles from aggregating inside the sticky liquid polymer. Instead, since the nanoparticles are easily suspending into the organic solvent, it utilizes the organic solvent and sonication to suspend the particles. This method can take one hour or less to prepare the nanoparticles suspension.

With this method, a magnet can be used to attract the nanoparticles into the microstructures on the mold. The magnet can attach closely to the mold, and the magnet can be circled around the center of the microstructure region. This step can be continued until the about 70% of the organic solvent evaporates. The deeper the microstructures, the longer time of circling is to remove the same amount of the organic solvent. The magnet can be moved quickly out of the microstructure region, during which the magnet can remain attached to the opposite side of the mold. This step can remove excess nanoparticles outside the microstructures while the nanoparticles inside the microstructures stay there.

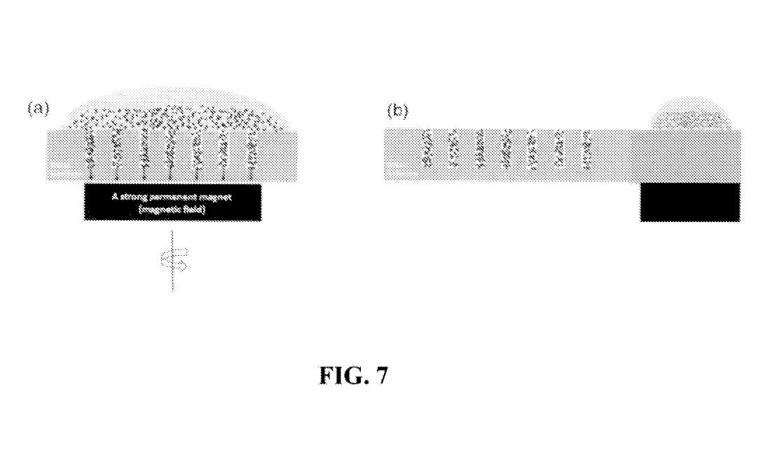

The illustration of one way of using magnet is illustrated in the FIG. 7. In FIG. 7(a), a magnet was attached to the back side of a mold and can be kept circling around the center of the microstructure region. The deeper the microstructures, the longer the circling of the magnet. FIG. 7(b) shows that after the circling process, when the magnet was moved out of the microstructure region, excess nanoparticles were removed out of the microstructure region with the movement of the magnet.

### Properties of Fe Nanoparticle Filled Micropillars

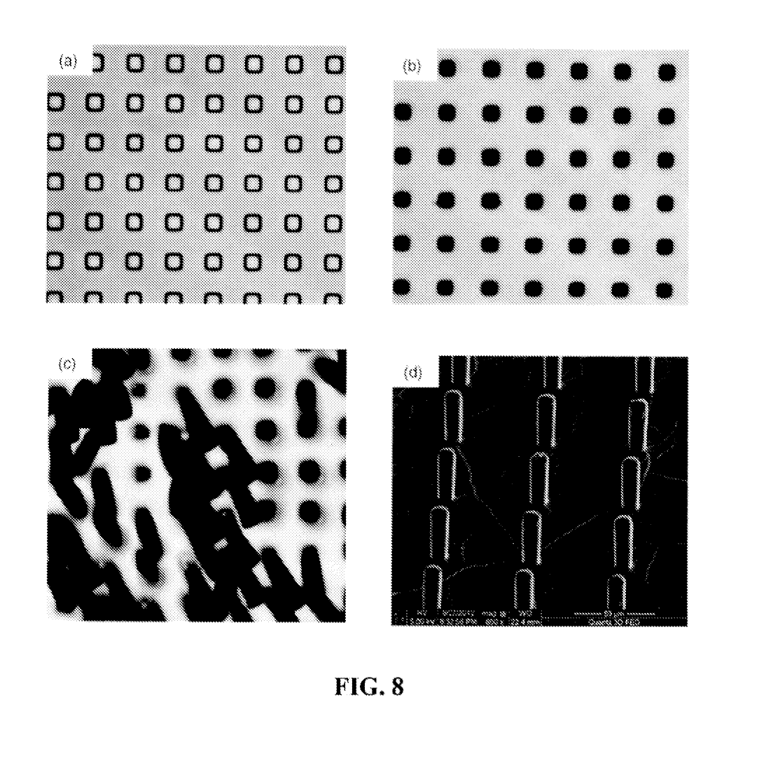

The transmitted light microscopic images of fabricated PDMS micropillar array are shown in FIG. 8. FIG. 8(a) shows a microscope image of PDMS micropillar array without Fe nanoparticles. FIG. 8(b) shows a microscope image of PDMS micropillar array filled with Fe nanoparticles (composite pillar). FIG. 8(c) shows a microscope image of the side view of scratched down composite pillars. FIG. 8(d) shows scanning electron microscope (SEM) image of the composite pillar array.

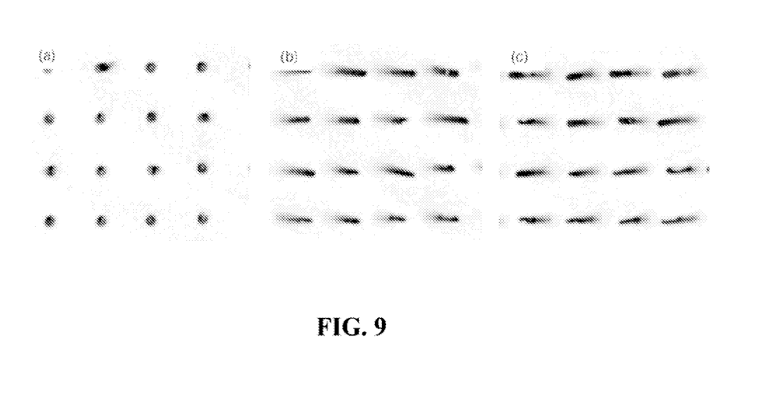

To test the displacement of the composite micropillar array under a magnetic field, the film of the pillar array was put at the bottom of a standard 100 mm plastic petridish and a 1″×0.75″ (D×H) Neodymium iron baron magnet was put on the top side of the petridish cover. The magnet was sliding back and forth manually on the petridish cover surface. The displacement of the pillar array was observed through a Zeiss Primo Vert Inverted microscope. The images indicating the movement of the composite pillar array under the magnetic force were shown in FIG. 9. The max displacement observed for a 3 μm size and 80 μm high pillar array was 13 μm.

Pillar size is 3 μm in FIG. 9. FIG. 9(a) shows a microscope image of polymer pillar array with no magnet applied; FIG. 9(b) shows the displacement of polymer pillar array when magnet was applied to the top right of the pillar array; and FIG. 9(c) shows the displacement of polymer pillar array when magnet was applied to the top left of the pillar array.

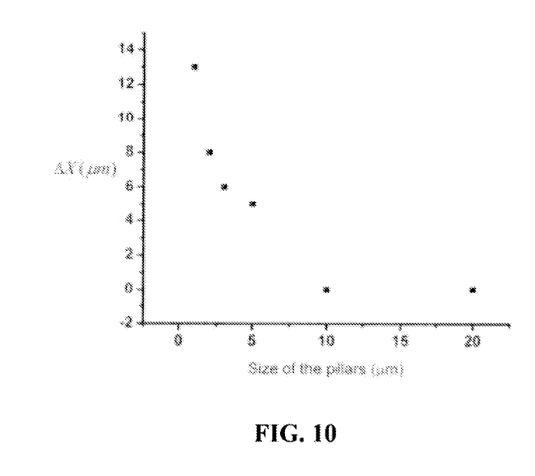

Polymer pillar arrays with 60 μm height and sizes of 1 μm, 2 μm, 3 μm, 5 μm, 10 μm, and 20 μm were fabricated using the above described method. And the displacements of these different sized pillars were measured with the results shown in FIG. 10.

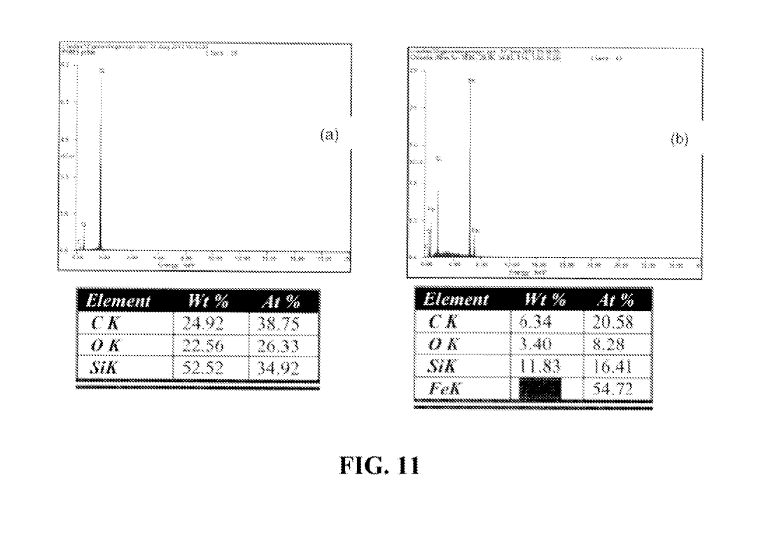

The percentage of the Fe nanoparticles powder contained inside the pillars was measured using SEM with EDX attachment. The result in FIG. 11b shows that the percentage can be as high as 78%. FIG. 11a shows the EDX measurement of PDMS pillar array without Fe nanoparticle as a comparison.

### “Shaking Bed” Nematode Study Device Using Fe Nanoparticle Filled Micropillars

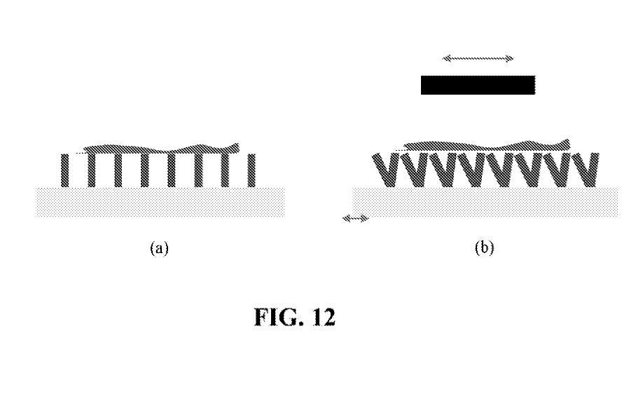

FIG. 12 is a schematic drawing of a nematode study device. In FIG. 12(a), nematode was loaded onto Fe nanoparticles filled pillar array; the pillar array was on a PDMS substrate. FIG. 12(b) shows the deformation of the pillars deformed with the lateral movement of an external magnet.

Once nematode is loaded onto the Fe nanoparticles filled pillar array, a magnet will be added externally to attract the pillars move toward the magnet position. The magnet will be moved into different position in a constant or varied frequency. Then the pillars on the pillar array will deform with the move of the magnet and thus provide a mechanical stimulation to the nematode on the pillar array. The study of the response of the nematode under such mechanical stimulation can provide more insight to the biological information of nematode.

### Cell Mechanics Study Device Using Fe Nanoparticle Filled Micropillars

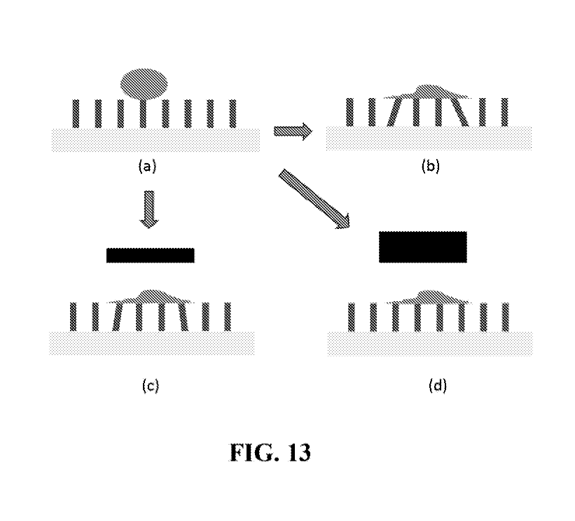

The setup of the device is shown in FIG. 13. Cells are loaded onto these devices, and then magnet will be added vertically to the pillar array. When cells begin to adhere to the top surface of pillars, cells can exert traction force to the pillars underneath them and this will lead to the deformation of the pillars towards the cell body if external magnet is not applied. But with the external magnet, the deformation of the pillar will be limited or even overcome by the magnetic force. With different field strength magnet, the degree of overcome of the pillar deformation will be different. Such overcome force can be sensed by the cells and cells may modify their morphology to adapt to such overcome force. Such modification of the cell morphology may lead to complicated biological change within cells and so will affect cell adhesion, signaling and proliferation. Thus the Fe nanoparticles filled PDMS pillar array can be used as the cell mechanics study device.

In FIG. 13, the substrate is PDMS substrate. FIG. 13(a) is a schematic drawing of loading cells onto a pillar array; FIG. 13(b) is a schematic drawing showing that cell spreading on and adhesion to the top of the pillars cause the deformation of the underneath pillars toward the center of the cell body; FIG. 13(c) is a schematic drawing showing that when applying a magnetic force by adding a magnet above the pillar array, the deformation of the pillars underneath the cells is reduced; FIG. 13(d) is a schematic drawing showing that when a strong magnetic force is applied, the magnetic force can overcome the traction force of cell and can prevent the deformation of the pillars underneath the cells.

### Microfluidic Micromixer Using Fe Nanoparticle Filled Micropillars

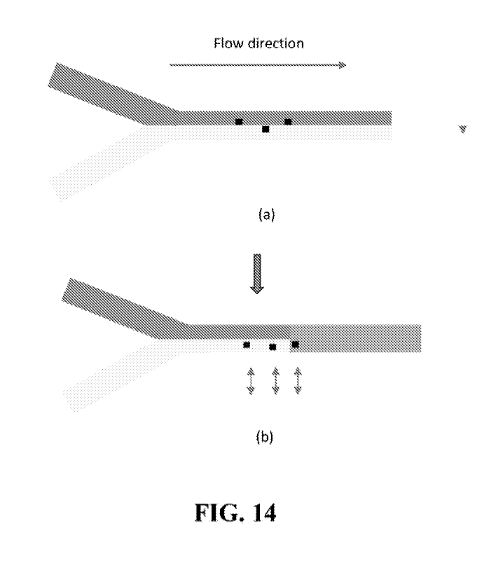

The setup of the device is shown in FIG. 14. When two different kinds of liquid were injected into a channel, the nanoparticles filled pillar can swing or swirl due to external magnetic force to mix the liquid. Micromixer is an important component for microfluidic device.

FIG. 14(a) is a schematic drawing showing that when two liquids (light color and dark color indicate two types of liquids) are injected into a Y junction microfluidic channel, if no external magnetic force is exerted, liquid/liquid interface is observed in the downstream of the Y junction channel; and FIG. 14(b) is a schematic drawing showing that with the application of external magnetic force, the pillars (the dots in the channel) can move or swirl vertically to the flow direction and thus cause mixture of two kinds of liquid in the downstream of the Y junction channel.

Other embodiments are within the scope of the following claims.

What is claimed is:

1. A three-dimensional microstructure comprising a plurality of collapsed polymer columns, wherein a portion of a surface of each collapsed polymer column contacts with a portion of a surface of at least another collapsed polymer column.

2. The three-dimensional microstructure of claim 1, wherein the surface of each collapsed column includes nanoscale grooves.

3. The three-dimensional microstructure of claim 1, wherein at least two collapsed polymer columns have substantially similar height and size dimensions when straightened.

4. The three-dimensional microstructure of claim 1, wherein at least two collapsed polymer columns have substantially different height and size dimensions when straightened.

5. The three-dimensional microstructure of claim 1, wherein the size of a collapsed polymer column is less than 7 μm.

6. The three-dimensional microstructure of claim 1, wherein the size of a collapsed polymer column is less than 5 μm.

7. The three-dimensional microstructure of claim 1, wherein the size of a collapsed polymer column is less than 2 μm.

8. The three-dimensional microstructure of claim 1, wherein the three-dimensional microstructure is within a microwell.

9. The three-dimensional microstructure of claim 1, wherein each collapsed polymer column includes polydimethylsiloxane, poly(methyl methacrylate), epoxy resin, polyethylene glycol, or a copolymer thereof.

10. A device for culturing cells comprising a three-dimensional microstructure,
wherein the three-dimensional microstructure comprising:
a plurality of collapsed polymer columns, wherein a portion of a surface of each collapsed polymer column contacts with a portion of a surface of at least another collapsed polymer column.

11. The device of claim 10, wherein the surface of each collapsed column includes nanoscale grooves.

12. The device of claim 10, wherein at least two collapsed polymer columns have substantially similar height and size dimensions if straightened.

13. The device of claim 10, wherein at least two collapsed polymer columns have substantially different height and size dimensions if straightened.

14. The device of claim 10, wherein the size of a collapsed polymer column is less than 7 μm.

15. The device of claim 10, wherein the size of a collapsed polymer column is less than 5 μm.

16. The device of claim 10, wherein the size of a collapsed polymer column is less than 2 μm.

17. The device of claim 10, wherein the three-dimensional microstructure is within a microwell.

18. The device of claim 10, wherein each collapsed polymer column includes polydimethylsiloxane, poly(methyl methacrylate), epoxy resin, polyethylene glycol, or a copolymer thereof.

19. The device of claim 10, wherein the cells include a HeLe cell, a SaOs-2 cell, or a NIH-3T3 cell.

20. The device of claim 10, wherein the cells are viable.

21. The device of claim 10, wherein the cells are on and within the three-dimensional microstructure.

22. The device of claim 10, wherein the cells are on the nanoscale grooves of the collapsed columns.

23. A three dimensional microstructure comprising a plurality of polymer pillars on the surface of a substrate, wherein each polymer pillar includes a plurality of nanoparticles.

24. The three dimensional microstructure of claim 23, wherein the polymer includes polydimethylsiloxane, poly(methyl methacrylate), epoxy resin, polyethylene glycol, or a copolymer thereof.

25. The three dimensional microstructure of claim 23, wherein the nanoparticles include magnetic nanoparticles.

26. The three dimensional microstructure of claim 23, wherein the nanoparticles include iron, cobalt, nickel, a compound thereof, or an alloy thereof.

27. The three dimensional microstructure of claim 23, wherein the nanoparticles include iron nanoparticles.

28. The three dimensional microstructure of claim 23, wherein the size of a polymer pillar is less than 20 μm.

29. The three dimensional microstructure of claim 23, wherein the size of a polymer pillar is less than 10 μm.

30. The three dimensional microstructure of claim 23, wherein the size of a polymer pillar is less than 5 μm.

31. The three dimensional microstructure of claim 23, wherein the height of a polymer pillar is less than 100 μm.

32. The three dimensional microstructure of claim 23, wherein the height of a polymer pillar is less than 50 μm.

33. The three dimensional microstructure of claim 23, wherein the height of a polymer pillar is less than 20 μm.

34. A device comprising:
a plurality of polymer pillars, wherein each polymer pillar includes magnetic nanoparticles;
a magnetic field source that deforms the polymer pillars when applied to the pillars.

35. The device of claim 34, wherein the magnetic field source operates at a range of magnetic field strength.

36. The device of claim 34, wherein the deformation of the polymer pillars provides a mechanical stimulation to a subject on the polymer pillar.

37. The device of claim 36, wherein the subject is a worm.

38. The device of claim 36, wherein the subject is a cell.

39. A device for mixing liquid comprising:
a Y junction microfluidic channel wherein at least two different kinds of liquid can be separately inserted into the channel;
a plurality of polymer pillars within the channel, wherein each polymer pillar includes magnetic nanoparticles; and
a magnetic field that operates at a range of magnetic field strength, wherein the liquid is mixed by applying a magnetic field to the polymer pillars.
